(19) United States
(12) Patent Application Publication
(10) Pub. No.: US 2026/0364223 A1
(43) Pub. Date: Nov. 19, 2026

(54) INDUCTIVE UNLOCKING ATOMIZER

(71) Applicant: AROMA PERFECTO LLC, Diamond Bar, CA (US)

(72) Inventor: Yong WU, Guangzhou (CN)

(73) Assignee: AROMA PERFECTO LLC, Diamond Bar, CA (US)

(21) Appl. No.: 19/458,606

(22) Filed: Jan. 23, 2026

(30) Foreign Application Priority Data
May 16, 2025 (CN) 202520976078.0

(51) Int. Cl. A61L 9/14; F24F 8/50; F24F 13/20; H01F 7/06

(52) U.S. Cl. CPC A61L 9/14; F24F 8/50; F24F 13/20; H01F 7/064; A61L 2209/133

(57) ABSTRACT

An inductive unlocking atomizer includes a housing, an atomizing assembly positioned in the housing, a locking mechanism configured for locking the atomizing assembly to the housing, and a separate unlocking part configured for releasing locking of the locking mechanism via induction. The housing or the atomizing assembly is provided with an unlocking area for supporting the unlocking part during unlocking, and the locking mechanism is configured to unlock the atomizing assembly from the housing, in response to the unlocking part being on or within the unlocking area, and further configured to relock the atomizing assembly to the housing, in response to the unlocking part being moved away from the unlocking area.

### CROSS-REFERENCE TO RELATED APPLICATIONS

The present application claims priority to Chinese Patent Application No. 202520976078.0, filed on May 16, 2025, the content of which is incorporated herein by reference in its entirety .

### TECHNICAL FIELD

The present disclosure relates to the field of atomizers, and more particularly, to an inductive unlocking atomizer.

### BACKGROUND

In the technical field of aroma diffusers, particularly in the design of commercial and household aroma diffusion equipment, the convenient replacement of aroma liquid and the safety of the equipment have always been focus points in research and development. To achieve quick replacement of the aroma liquid, existing aroma diffusers generally have a modular design, allowing both an atomizing assembly and an aroma liquid container to be detachable from an aroma diffuser body. This design not only facilitates users to change different scents of aroma liquid according to their needs but also makes the equipment easier to clean and maintain.

Additionally, to prevent the aroma components from being stolen in commercial environments and to prevent children from maloperation or ingesting the aroma liquid in household environments, the existing aroma diffusers are typically equipped with locking devices. These locking devices serve to securely lock the atomizing assembly, and the aroma liquid container, and the aroma diffuser body together. This effectively enhance the safety of the equipment.

However, although these locking devices meet safety requirements to a certain extent, they are often structurally complex and cumbersome to operate. These locking devices usually require a special key to open, which not only increases the burden on users to carry the key but also has higher potential for losing the key, causing inconvenience to the users. Especially in commercial places where a large number of aroma diffusers exist, managing the keys becomes a tedious task, increasing management costs and potentially causing the equipment to be unusable due to confusion or loss of key.

To overcome the above-mentioned problems, for example, Chinese Patent Publication CN217908442U provides a fragrance-diffusing equipment having a novel locking structure, which can achieve detachable fixation of an atomizing head with simplified locking mechanism. Particularly, the locking structure includes a pin and a slot which are respectively arranged on the equipment body and the atomizing head. The pin can be driven by an electromagnetic mechanism built in the equipment body or by an external magnetic mechanism to enter or exit the slot, so as to achieve locking or unlocking operation. This design eliminates the need for complex mechanical structures and special keys, significantly simplifying the structure of the locking device. However, the locking mechanism in such configuration is placed on the side of the equipment body, which requires the magnetic unlocking part to be firmly attached to the vertical side wall of the body for unlocking. As a result, even with markings, it is not easy to find the exact unlocking position, which often leads to failure to unlock, causing inconvenience in use.

Therefore, how to ensure the safety of the aroma diffuser while simplifying the structure of the locking device, enhancing convenience of user operation, and reducing trouble of key management has become an urgent technical problem to be solved in the design of aroma diffusers.

### SUMMARY

It is therefore an object of the present disclosure to provide an inductive unlocking atomizer that is quick and convenient to unlock.

The inductive unlocking atomizer of the present disclosure includes a housing, an atomizing assembly positioned in the housing, a locking mechanism for locking the atomizing assembly to the housing, and a separate unlocking part for releasing locking of the locking mechanism via induction. In addition, the housing or the atomizing assembly is provided with an unlocking area for supporting the unlocking part during unlocking. The locking mechanism is configured in such a way that when the unlocking part is placed on or within the unlocking area, the locking between the atomizing assembly and the housing is released, and when the unlocking part is moved away from the unlocking area, the locking mechanism restores the locking between the atomizing assembly and the housing.

The unlocking area of the present disclosure not only serves to support the unlocking part but also ensures accurate positioning of the unlocking part. In order to achieve accurate association between the unlocking part and the locking mechanism, it only requires the users to place the unlocking part on or within the unlocking area, which not only frees up hands of the user to easily disassemble the atomizing assembly from the housing for aroma liquid replacement, but also prevents maloperations. The inductive unlocking atomizer of the present disclosure is especially convenient for mounting in special positions, such as in high or narrow spaces, which also improves the user experience with the hands not occupied during disassembling.

The inductive unlocking atomizer of the present disclosure may be applicable to various locking mechanisms, for example the unlocking between the unlocking part and the locking mechanism may be achieved by at least one of magnetic unlocking, radio frequency identification (RFID) unlocking, or Hall sensing unlocking. Generally, the mechanic locking mechanisms uses the magnetic unlocking, while the electronic locking mechanism uses the RFID unlocking or the Hall sensing unlocking.

To reduce costs and energy consumption, the unlocking between the unlocking part and the locking mechanism may be preferably achieved by the magnetic unlocking. For this purpose, the unlocking area in a preferable embodiment is particularly in form of a flat surface or a recess on the housing or the atomizing assembly for supporting the unlocking part, and the locking mechanism is installed adjacent to the unlocking area to ensure mutual attraction with the unlocking part. The flat surface area or recess can facilitate quick positioning of the unlocking part, and also support the unlocking part to prevent it from falling or shifting.

In an alternative embodiment, the unlocking area may protrude from an upper surface of the housing. This allows the unlocking part to be readily removed from the unlocking area.

According to a preferable embodiment of the present disclosure, the locking mechanism may particularly include a magnetic actuator and a locking actuator, and the locking actuator is driven by interaction between the unlocking part and the magnetic actuator to release the locking of the locking mechanism.

Furthermore, the locking actuator may be an elastic pin structure arranged inside the housing or the atomizing assembly, and a locking hole associated with the pin structure may be correspondingly provided in the atomizing assembly or the housing. The locking actuator is inserted into the locking hole along a first axis to achieve locking between the atomizing assembly and the housing. During unlocking, the magnetic actuator is driven by the unlocking part to move along a second axis, so as to drive the locking actuator to disengage from the locking hole along the first axis. The second axis is not parallel to the first axis. Such configuration allows the magnetic actuator and the locking actuator to move in a non-parallel manner, for example the locking actuator arranged on the side of the housing achieves locking in a lateral direction, and the magnetic actuator is actuated at a certain angle relative to the locking actuator, which facilitates the unlocking area being placed on the top of the housing or the atomizing assembly, thereby allowing formation of the flat surface or recess for accommodating the unlocking part.

To make the overall structure more concise or compact, the second axis may be preferably perpendicular to the first axis. For this purpose, the locking actuator according to a preferable embodiment particularly includes a pin and an elastic element, where the pin is pushed by the elastic element to be inserted into the locking hole along the first axis, and the pin is provided with a first inclined surface. The magnetic actuator in this case particularly includes a support frame, a sliding frame, and an attracted end, where the sliding frame is slidably installed inside the support frame along the second axis and passes through the support frame to connect with the attracted end. The sliding frame is provided with a second inclined surface. The pin is extended through the sliding frame to allow the first inclined surface to be abutted against the second inclined surface, such that when the sliding frame slides along the second axis, the second inclined surface can push the first inclined surface to drive the pin to compress the elastic element along the first axis. Such configuration is simple in structure, with high stability and reliable unlocking efficiency.

To further enhance the unlocking efficiency, the unlocking part in an advantageous embodiment has a magnetic attracting end, which includes a permanent magnet. The attracted end of the locking mechanism is also magnetic, which includes another permanent magnet. When the unlocking part is placed in the unlocking area, the attracting end can drive the attracted end to move vertically along the second axis, especially upward, to release locking of the locking mechanism. The permanent magnet on the locking mechanism can enhance the attraction force between the permanent magnet of the unlocking part. The combination of the permanent magnet on the locking mechanism and the vertical upward unlocking action allows the permanent magnet on the locking mechanism to push the locking mechanism to reset under the action of gravity, when the attracted end loses the attraction force from the attracting end, namely the unlocking part is moved away from the unlocking area, thereby achieving relocking.

The atomizing assembly in the present disclosure may include an atomizing base and an aroma liquid container connected to the atomizing base. The aroma liquid container in this case is sealed inside the housing via the atomizing base, and the atomizing base is locked to the housing as a whole by the locking mechanism. By sealing and locking the aroma liquid container inside the housing with the atomizing base, the convenience of overall design and usage of a product can be improved.

The housing in the present disclosure may be divided into different functional areas, including a power chamber and an atomizing chamber that are isolated from each other. A power supply component, an air source component, and a control component are arranged in the power chamber. The atomizing assembly is arranged in the atomizing chamber. The air source component is extended into the atomizing chamber through an air supply tube to supply high-pressure gas to the atomizing assembly.

According to the present disclosure, by providing the unlocking area and the inductive unlocking part, the user can quickly release the locking of the locking mechanism by simply placing the unlocking part on or in the unlocking area, without complex mechanical operations or special keys. The design of the unlocking area not only facilitates positioning of the unlocking part, but also makes the operation more intuitive and simpler, especially used in high or narrow spaces, significantly improving operational convenience. In addition, the inductive unlocking atomizer of the present disclosure is simple in structure and easy to maintain, further reducing costs and maintenance difficulties.

Further advantages, features and potential applications of the present application may be gathered from the description which follows, in conjunction with the embodiments illustrated in the drawings.

### BRIEF DESCRIPTION OF THE DRAWINGS

FIG. 1 is a perspective view of an inductive unlocking atomizer according to an embodiment of the present disclosure.

FIG. 2 is a sectional view of the inductive unlocking atomizer in FIG. 1.

FIG. 3 is an enlarged view of portion A in FIG. 2.

FIG. 4 is a disassembly view of the inductive unlocking atomizer in FIG. 1.

FIG. 5 is an enlarged view showing an unlocking area of the inductive unlocking atomizer in FIG. 1.

FIG. 6 is a partially exploded view of the inductive unlocking atomizer in FIG. 1.

FIG. 7 is an enlarged view of portion B in FIG. 6.

FIG. 8 is an enlarged sectional view of a locking mechanism of the inductive unlocking atomizer in FIG. 2.

FIG. 9 is a disassembly view of the locking mechanism in FIG. 8.

FIG. 10 is an assembly view of the locking mechanism of FIG. 8 in a locking state.

FIG. 11 is an assembly view of the locking mechanism of FIG. 8 in an unlocking state.

FIG. 12 is a perspective view of the locking mechanism in FIG. 8.

FIG. 13 is an exploded view of the locking mechanism in FIG. 12.

FIG. 14 is a sectional view of the inductive unlocking atomizer in FIG. 1 in disassembly state.

FIG. 15 is an enlarged view of portion C in FIG. 14.

FIG. 16 is another perspective of the inductive unlocking atomizer in FIG. 1, with a housing removed.

FIG. 17 is a perspective view of an inductive unlocking atomizer according to another embodiment of the present disclosure, with some parts omitted.

FIG. 18 is a partially exploded view of the inductive unlocking atomizer in FIG. 17.

FIG. 19 is an exploded view of a locking mechanism of the inductive unlocking atomizer in FIG. 17.

FIG. 20 is a sectional view of the inductive unlocking atomizer in FIG. 17.

FIG. 21 is an enlarged view of portion D in FIG. 20.

FIG. 22 is an assembly view of the locking mechanism of the inductive unlocking atomizer of FIG. 17 in a locking state.

FIG. 23 is an assembly view of the locking mechanism of the inductive unlocking atomizer of FIG. 17 in an unlocking state.

FIG. 24 is a perspective view of an inductive unlocking atomizer according to another embodiment of the present disclosure, with some parts omitted.

FIG. 25 is a partially exploded view of the inductive unlocking atomizer in FIG. 24.

FIG. 26 is an exploded view of a locking mechanism of the inductive unlocking atomizer in FIG. 24.

FIG. 27 is a sectional view of the inductive unlocking atomizer in FIG. 24.

FIG. 28 is an enlarged view of portion E in FIG. 27.

FIG. 29 is an assembly view of the locking mechanism of the inductive unlocking atomizer of FIG. 24 in a locking state.

FIG. 30 is an assembly view of the locking mechanism of the inductive unlocking atomizer of FIG. 24 in an unlocking state.

FIG. 31 is a partially exploded perspective view of an inductive unlocking atomizer according to another embodiment of the present disclosure, with some parts omitted.

FIG. 32 is an exploded view of a locking mechanism of the inductive unlocking atomizer in FIG. 31.

FIG. 33 is a sectional view of the inductive unlocking atomizer in FIG. 31.

FIG. 34 is an enlarged view of portion H in FIG. 33.

FIG. 35 is a partially exploded perspective view of an inductive unlocking atomizer according to another embodiment of the present disclosure, with some parts omitted.

FIG. 36 is an exploded view of the inductive unlocking atomizer in FIG. 35.

FIG. 37 is a sectional view of the inductive unlocking atomizer in FIG. 35.

FIG. 38 is an enlarged view of portion F in FIG. 37.

FIG. 39 is an assembly view of the locking mechanism of the inductive unlocking atomizer of FIG. 35 in a locking state.

FIG. 40 is an assembly view of the locking mechanism of the inductive unlocking atomizer of FIG. 35 in an unlocking state.

FIG. 41 is a partially exploded perspective view of an inductive unlocking atomizer according to another embodiment of the present disclosure, with some parts omitted.

FIG. 42 is an exploded view of a locking structure of the inductive unlocking atomizer in FIG. 41.

FIG. 43 is a sectional view of the inductive unlocking atomizer in FIG. 41.

FIG. 44 is an enlarged view of portion G in FIG. 43.

FIG. 45 is an assembly view of the locking mechanism of the inductive unlocking atomizer of FIG. 41 in a locking state.

FIG. 46 is an assembly view of the locking mechanism of the inductive unlocking atomizer of FIG. 41 in an unlocking state.

FIG. 47 is a partially exploded perspective view of an inductive unlocking atomizer according to another embodiment of the present disclosure, with some parts omitted.

FIG. 48 is an exploded view of a locking structure of the inductive unlocking atomizer in FIG. 47.

FIG. 49 is an assembly view of a locking structure of the inductive unlocking atomizer in FIG. 47.

FIG. 50 is a sectional view of the inductive unlocking atomizer in FIG. 47.

FIG. 51 is an enlarged view of portion I in FIG. 50.

FIG. 52 is an assembly view of the locking mechanism of the inductive unlocking atomizer of FIG. 47 in a locking state.

FIG. 53 is an assembly view of the locking mechanism of the inductive unlocking atomizer of FIG. 47 in an unlocking state.

FIG. 54 is perspective view of an inductive unlocking atomizer according to another embodiment of the present disclosure, with an unlocking part moved away from an unlocking area.

FIG. 55 is another perspective view of the inductive unlocking atomizer in FIG. 54, with the unlocking part located in the unlocking area.

### DETAILED DESCRIPTION OF THE EMBODIMENTS

The accompanying drawings of the present disclosure are solely for illustrative purposes and should not be construed as limiting the present disclosure. To better illustrate the following embodiments, certain parts in the drawings may be omitted, enlarged, or reduced, and do not represent the actual size of a product. It is understandable for those skilled in the art that some well-known structures and their descriptions may be omitted in the drawings.

In the drawings, identical or similar features are labelled with the same reference numerals in different embodiments.

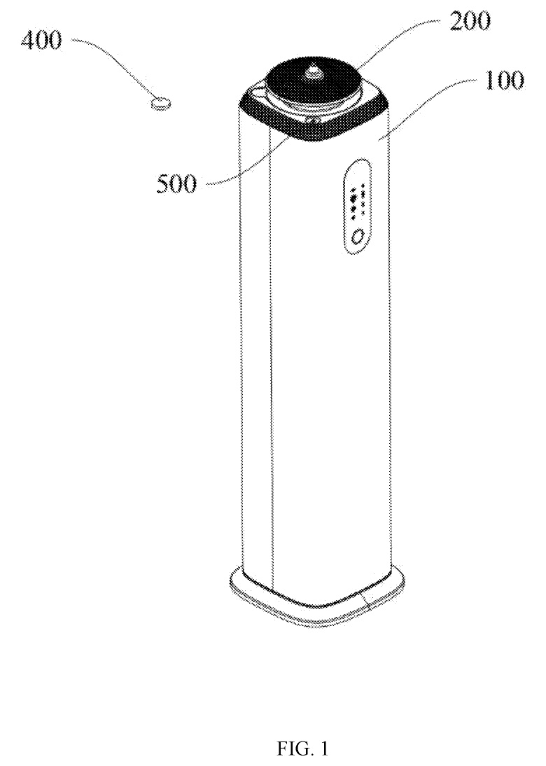

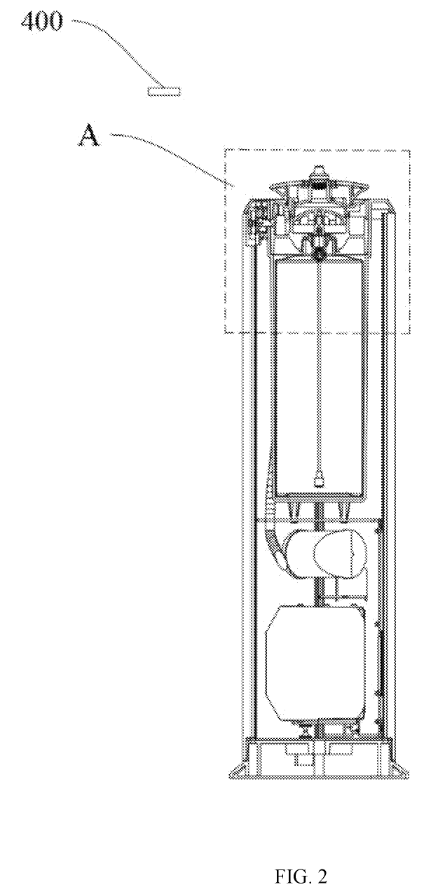

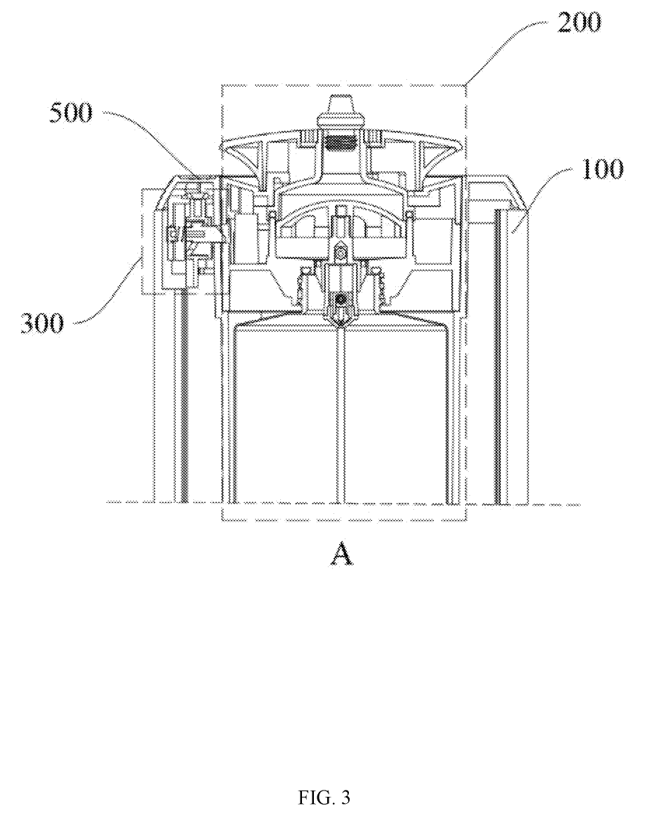

FIG. 1 provides an inductive unlocking atomizer according to an embodiment of the present disclosure, including a housing 100, an atomizing assembly 200, a locking mechanism 300 (not shown), and an unlocking part 400. The unlocking part 400 is an independent and separate component. As shown in FIGS. 2 and 3, the atomizing assembly 200 is installed in the housing 100 and locked to the housing 100 by the locking mechanism 300. The unlocking part 400 is configured to release, via induction, the locking imposed by the locking mechanism 300 between the atomizing assembly 200 and the housing 100.

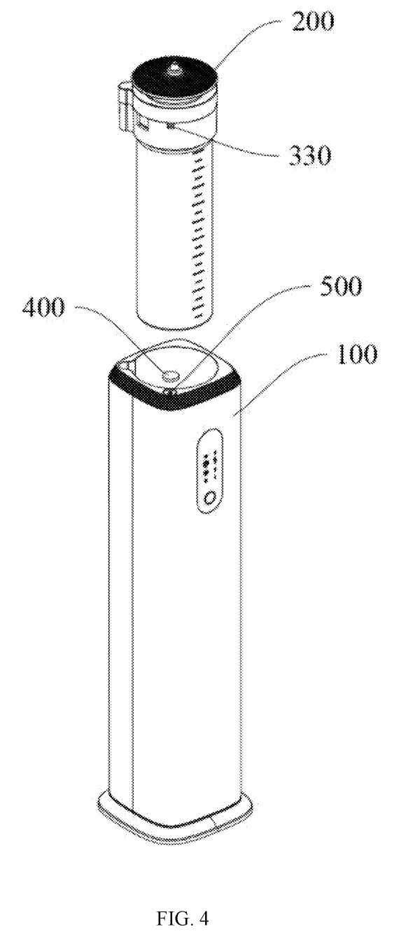

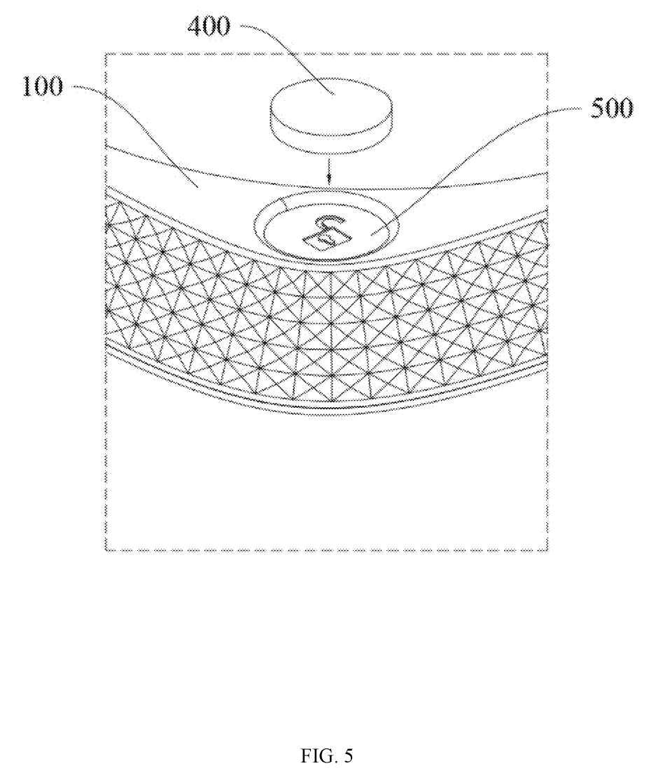

In combination with FIGS. 4 and 5, the housing 100 is provided with an unlocking area 500 for supporting the unlocking part 400 during unlocking. When the unlocking part 400 is placed in the unlocking area 500, the locking of the locking mechanism 300 can be released. As shown in FIGS. 1- 3, when the unlocking part 400 is moved away from the unlocking area 500, the locking mechanism 300 restores the locking between the atomizing assembly 200 and the housing 100.

Particularly, in this embodiment, the unlocking between the unlocking part 400 and the locking mechanism 300 is achieved through magnetic unlocking. It should be understood that, in other embodiments, other common inductive unlocking methods such as Hall sensing unlocking can also be employed. The unlocking area 500 in this embodiment is particularly a recess formed on the housing 100 for supporting the unlocking part 400 during unlocking. The locking mechanism 300 is installed adjacent to the unlocking area 500 to ensure mutual attraction with the unlocking part 400.

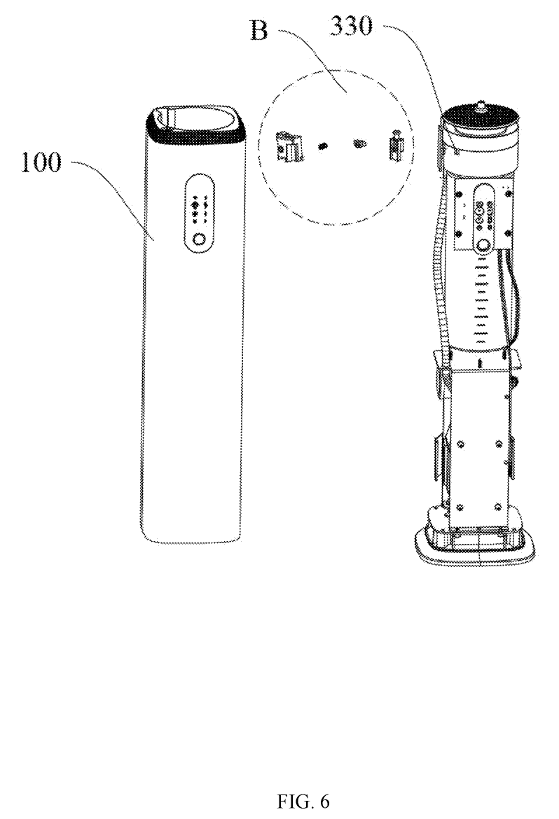

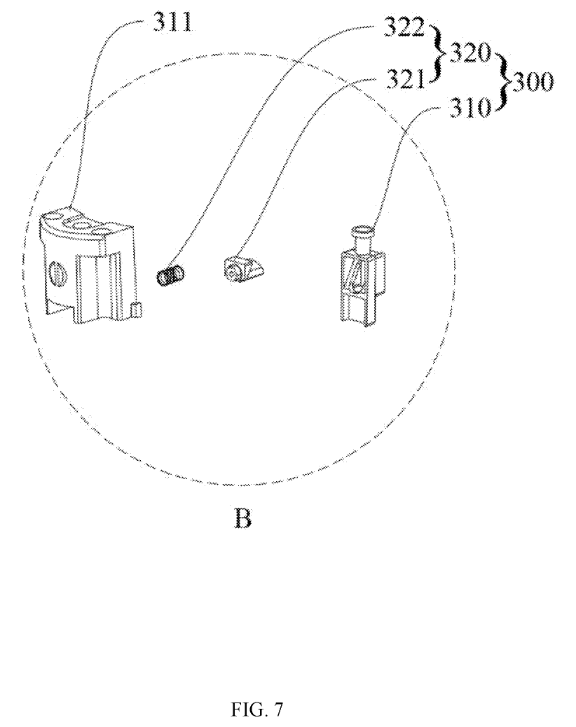

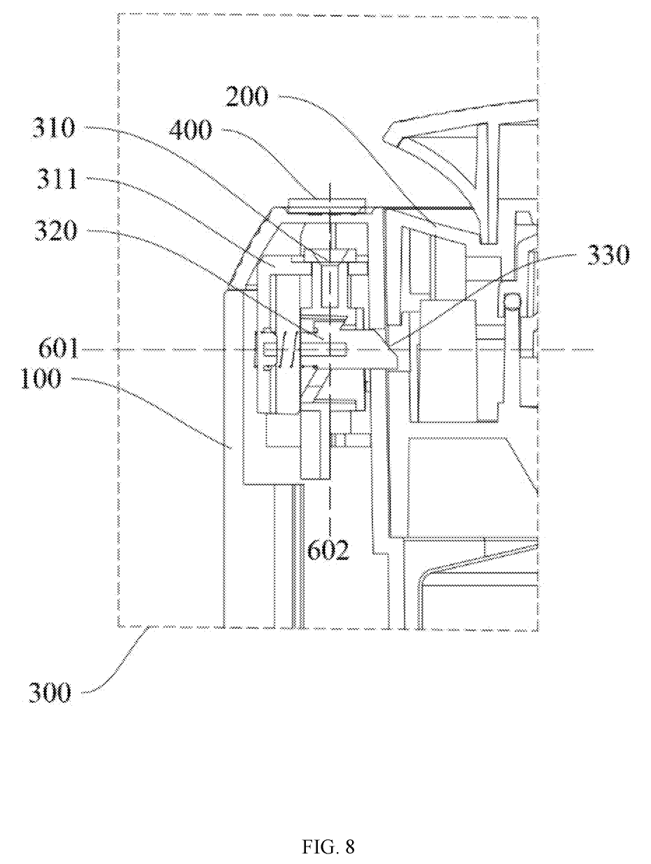

FIGS. 6 and 7 illustrate specific structure of the locking mechanism 300 according to an embodiment of the present disclosure. The locking mechanism 300 in this embodiment includes a magnetic actuator 310 and a locking actuator 320. In conjunction with FIG. 8, the locking actuator 320 is an elastic pin structure arranged inside the housing 100, which is associated with a locking hole 330 provided in the atomizing assembly 200. The unlocking part 400 cooperates with the magnetic actuator 310 to drive the locking actuator 320 to perform the unlocking action.

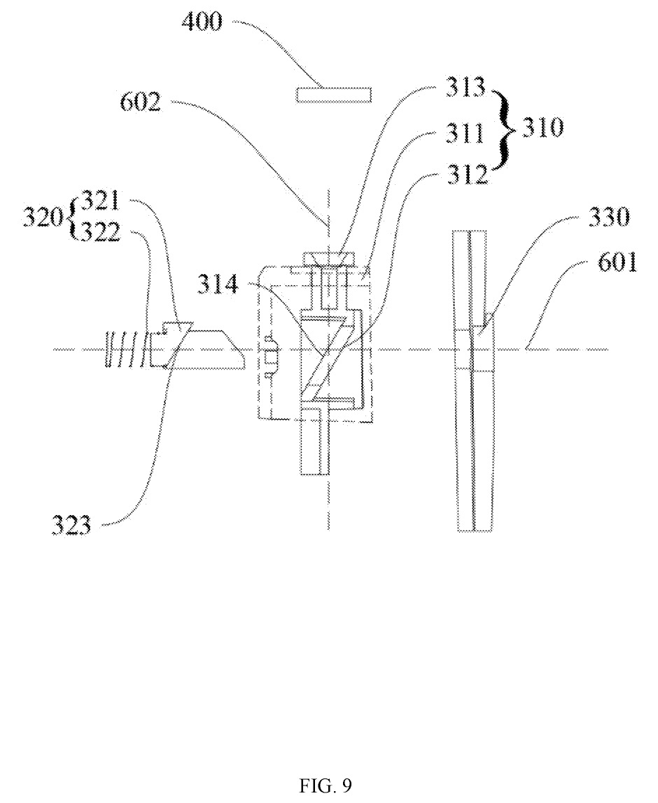

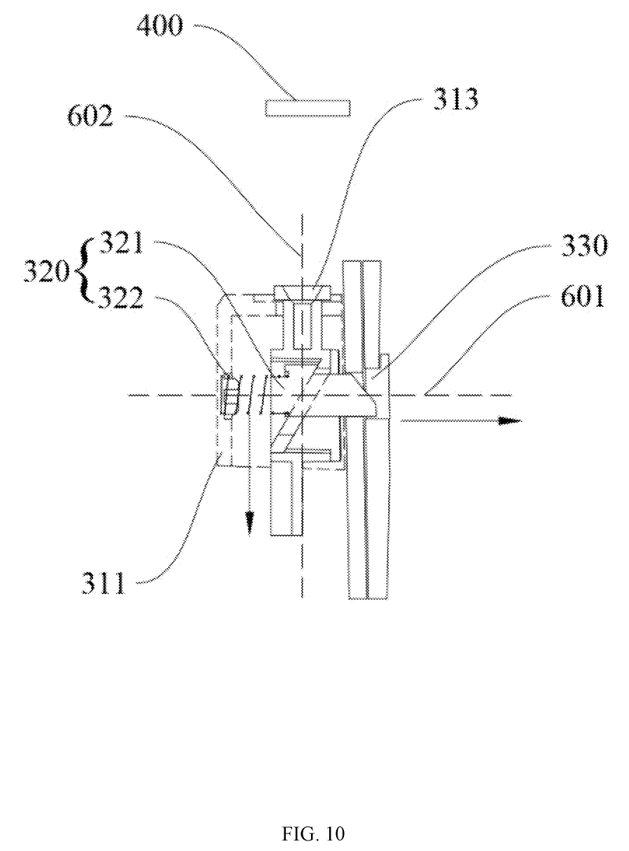

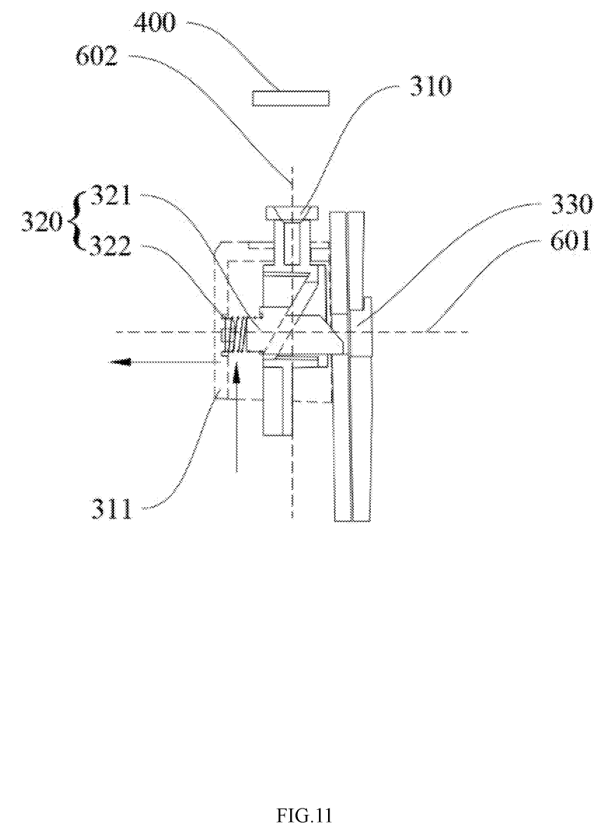

Referring to FIGS. 9 and 10, the locking actuator 320 is inserted into the locking hole 330 along a first axis 601 to achieve locking between the atomizing assembly 200 and the housing 100. Further referring to FIG. 11, the magnetic actuator 310 is driven by the unlocking part 400 to move along a second axis 602, so as to drive the locking actuator 320 to disengage from the locking hole 330 along the first axis 601, thus achieving the unlocking action. The second axis 602 in this embodiment is perpendicular to the first axis 601. It should be understood that the second axis 602 may not be perpendicular to the first axis 601, and an adaptable angle therebetween can be selected to match the actual fitting mode of the housing 100 and the atomizing assembly 200.

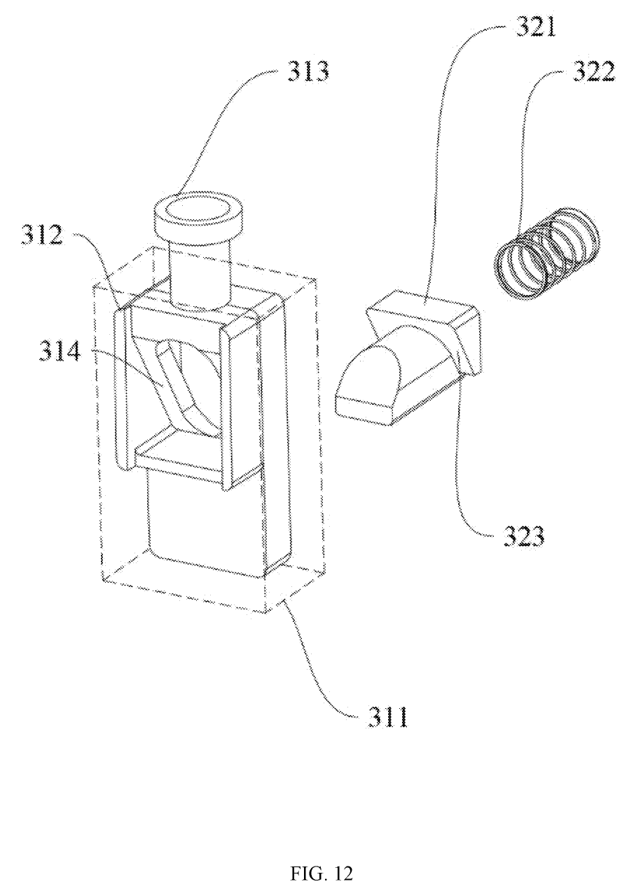

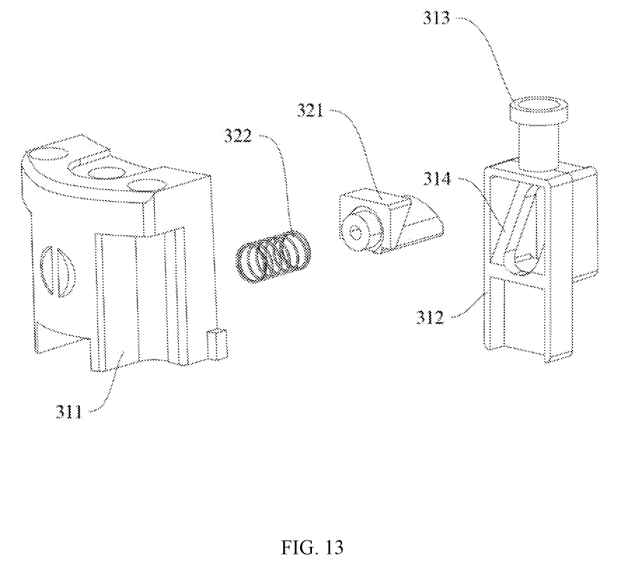

Further in combination with FIGS. 12 and 13, the locking actuator 320 in this embodiment includes a pin 321 having a first inclined surface 323, and an elastic element 322. The magnetic actuator 310 includes a support frame 311 having a second inclined surface 314, a sliding frame 312, and an attracted end 313. As illustrated in FIGS. 10 and 11, the elastic element 322 pushes the pin 321 to insert into the locking hole 330 along the first axis 601. The sliding frame 312 is slidably installed inside the support frame 311 along the second axis 602, with one end extending through the support frame 311 to connect with the attracted end 313. The pin 321 is extended through the sliding frame 312 to allow the first inclined surface 323 to be abutted against the second inclined surface 314, such that when the sliding frame 312 slides along the second axis 602, the second inclined surface 314 can push the first inclined surface 323 to drive the pin 321 to compress the elastic element 322 along the first axis 601.

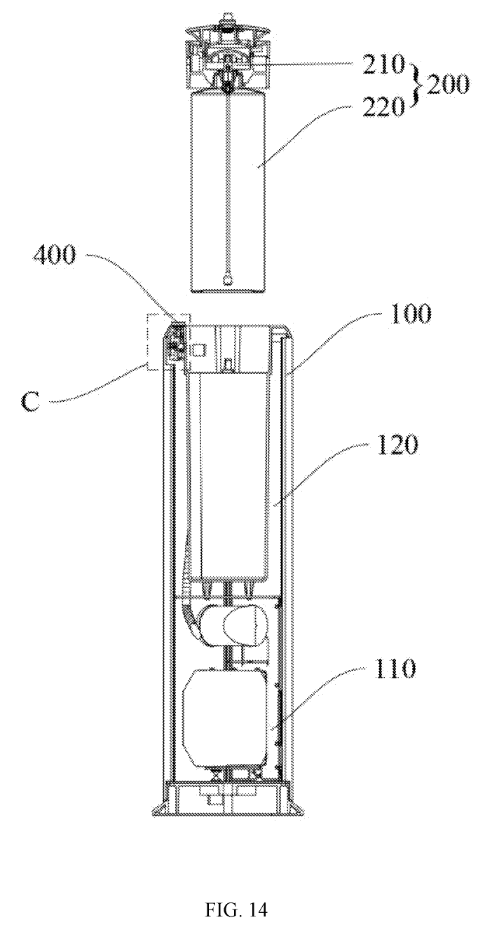

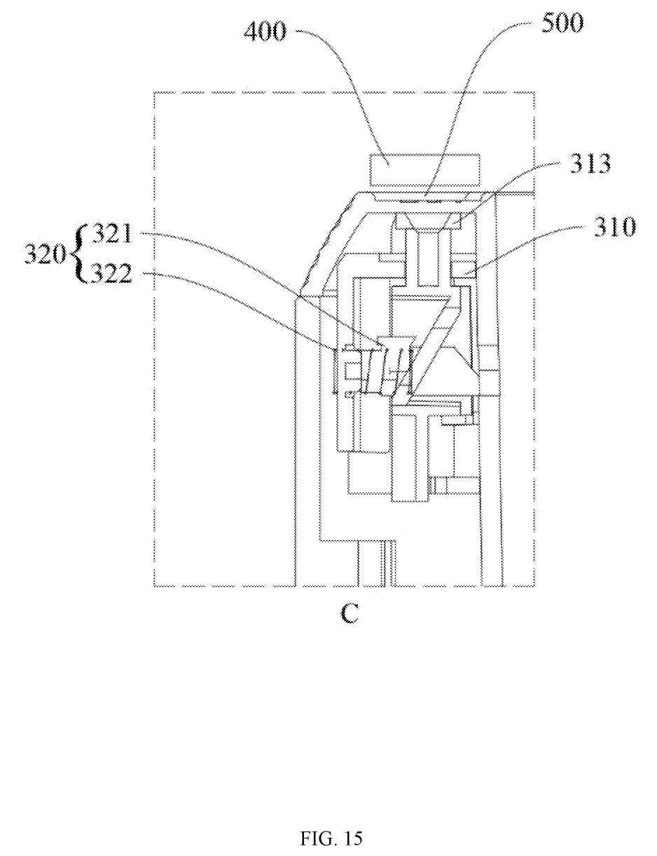

Now referring to FIGS. 14 and 15, the unlocking part 400 has a magnetic attracting end, which is a permanent magnet. The attracted end 313 of the locking mechanism 300 is also magnetic, which is another permanent magnet. When the unlocking part 400 is placed in the unlocking area 500, the attracting end of the unlocking part 400 drives the attracted end 313 to move vertically upward to release the locking of the locking mechanism 300. The two permanent magnets offer a stronger mutual attraction force, ensuring reliability of the magnetic unlocking. In addition, the permanent magnet on the attracted end 313 also increases the weight of the attracted end 313, ensuring that when the permanent magnet on the attracted end 313 loses attraction force from the unlocking part 400, the sliding frame 312 can fall under sufficient gravity, thereby enhancing reliability of the relocking.

As shown in FIG. 14, the atomizing assembly 200 in this embodiment particularly includes an atomizing base 210 and an aroma liquid container 220 connected to the atomizing base 210. In conjunction with FIG. 2, the aroma liquid container 220 is sealed inside the housing 100 by the atomizing base 210, and the atomizing base 210 is locked to the housing 100 as a whole by the locking mechanism 300.

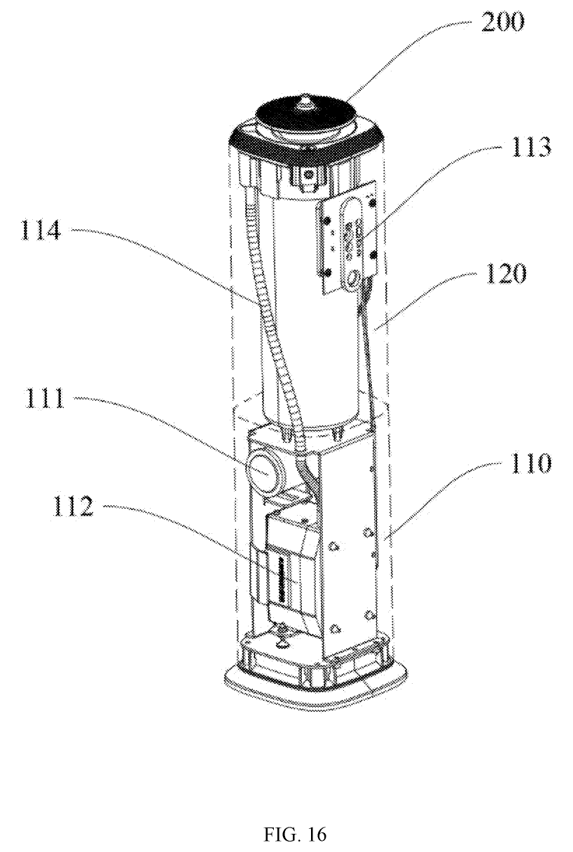

Further referring to FIG. 16, the housing 100 in this embodiment particularly includes a power chamber 110 and an atomizing chamber 120 that are isolated from each other. A power supply component 111 and an air source component 112 are arranged in the power chamber 110. The atomizing assembly 200 and a control component 113 are arranged in the atomizing chamber 120. The air source component 112 is extended into the atomizing chamber 120 through an air supply tube 114 to supply high-pressure gas to the atomizing assembly 200.

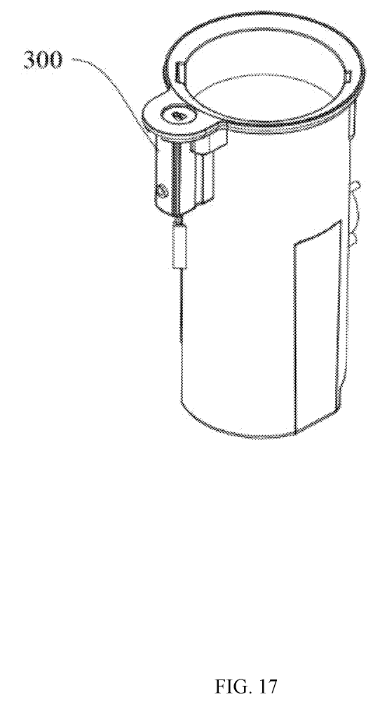

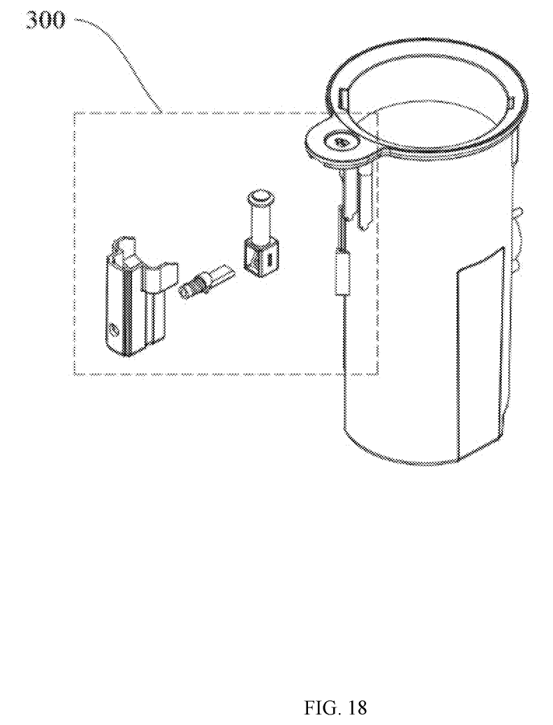

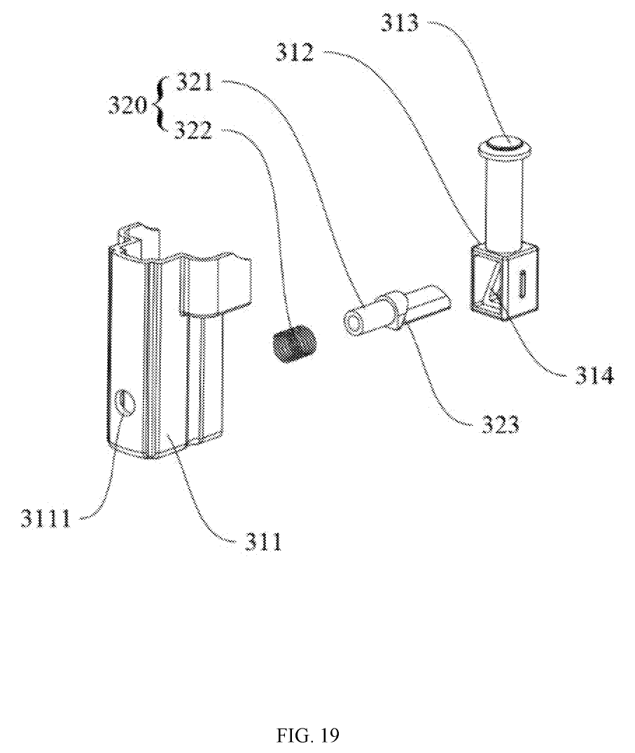

FIGS. 17 and 18 provide another locking mechanism 300 according to an embodiment of the present disclosure. As shown in FIG. 19, the locking actuator 320 in this embodiment includes a pin 321 and an elastic element 322. The magnetic actuator 310 includes a support frame 311, a sliding frame 312, and an attracted end 313 connected to the sliding frame 312. The support frame 311 in this embodiment is provided with a directional hole 3111 corresponding to the pin 321. The pin 321 is provided with a first inclined surface 323, and the sliding frame 312 is provided with a second inclined surface 314.

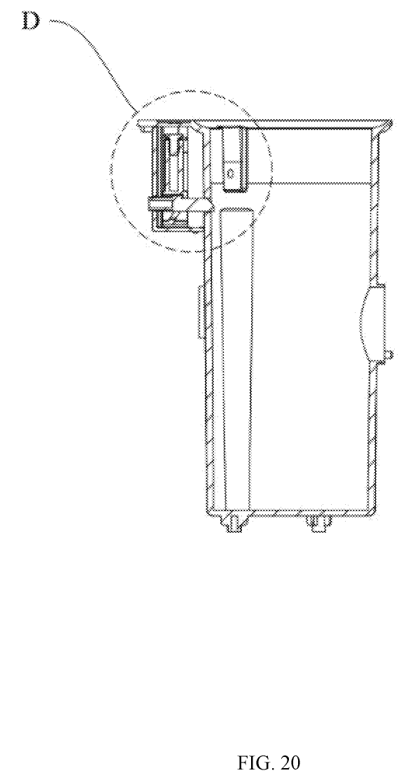

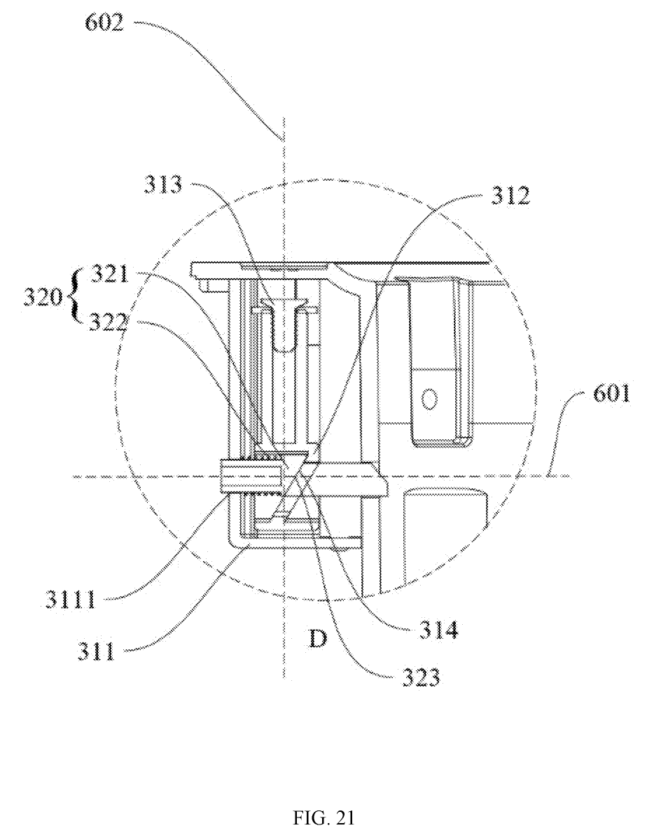

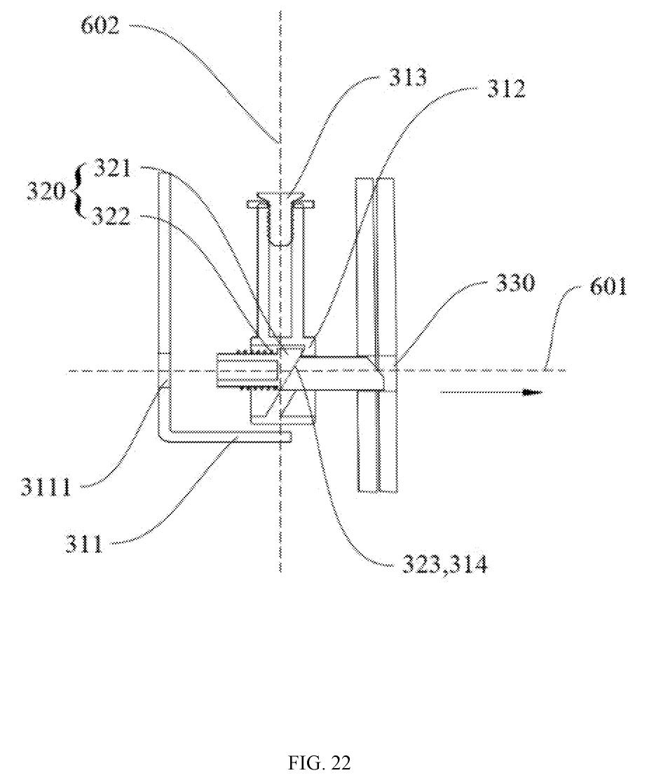

Referring to FIGS. 20, 21, and 22, the sliding frame 312 is slidably installed inside the support frame 311 along the second axis 602, and the elastic element 322 is sleeved on the pin 321. The end of the pin 321 facing the locking hole 330 is pushed by the elastic element 322 to pass through the sliding frame 312 and then be inserted into the locking hole 330 along the first axis 601, allowing the first inclined surface 323 to be abutted against the second inclined surface 314. The end of the pin 321 away from the locking hole 330 is extended into the directional hole 3111, which can slide in the directional hole 3111.

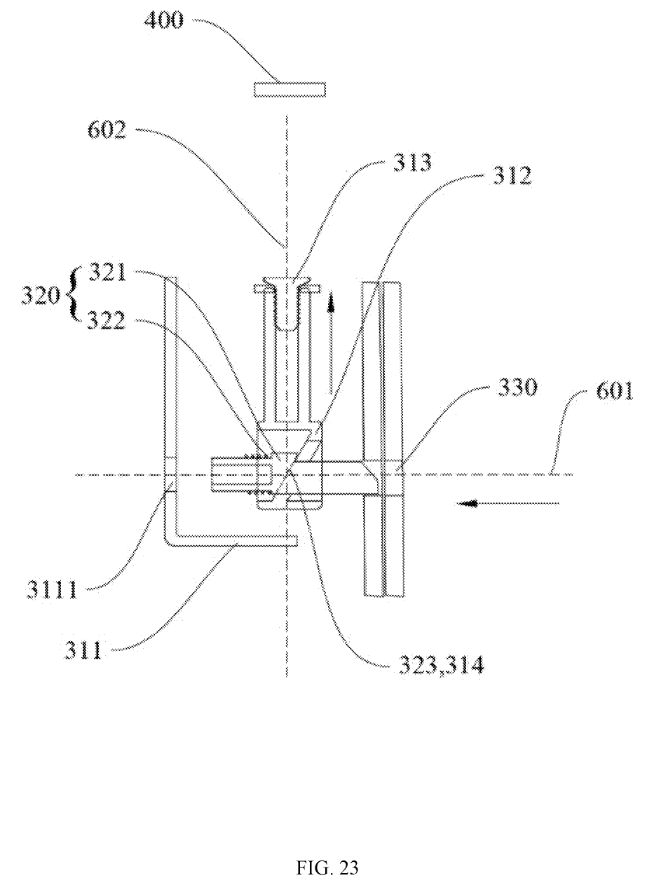

As shown in FIG. 23, when the unlocking part 400 is close to the unlocking area 500, or placed in the unlocking area 500, the sliding frame 312 slides along the second axis 602, allowing the second inclined surface 314 to push the first inclined surface 323 to drive the pin 321 to compress the elastic element 322 along the first axis 601, so as to drive the locking actuator 320 to disengage from the locking hole 330, thereby achieving the unlocking action.

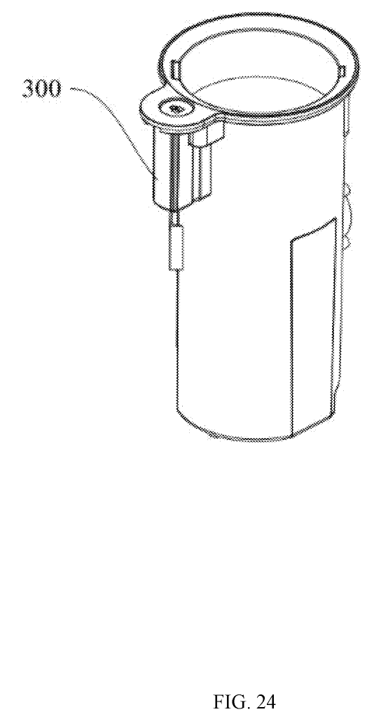

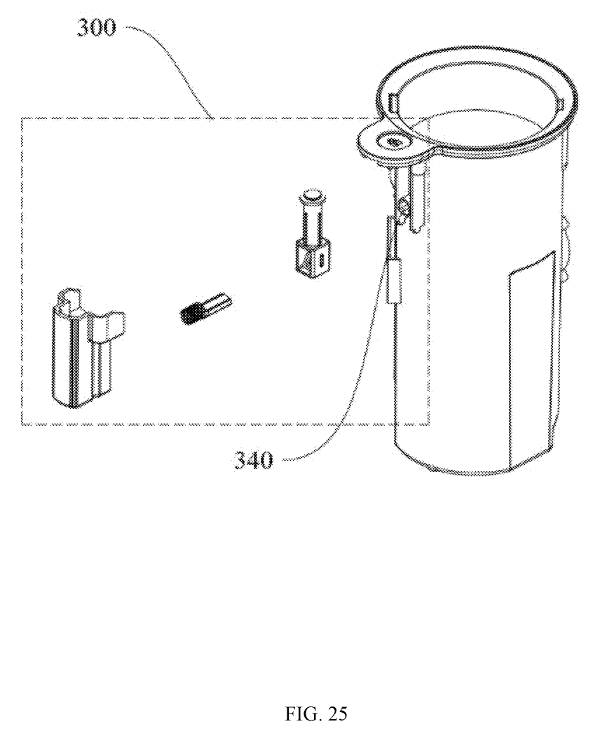

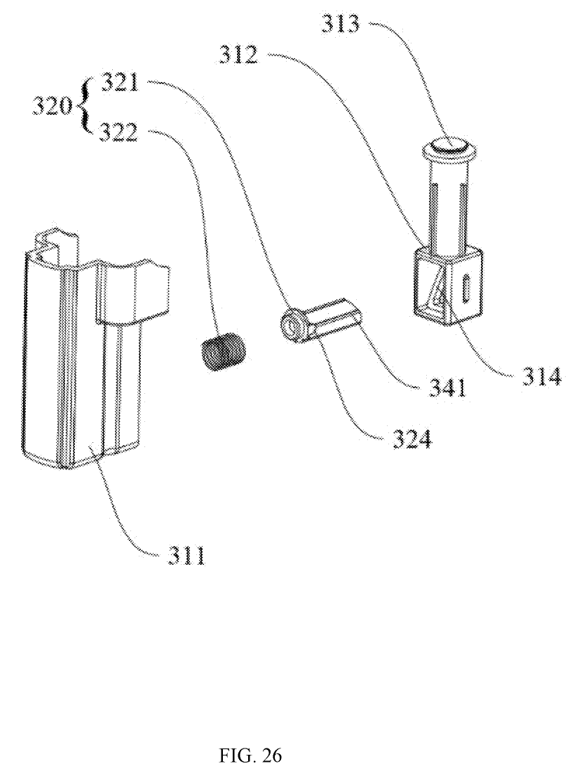

FIGS. 24 and 25 provide another locking mechanism 300 according to an embodiment of the present disclosure. As shown in FIG. 26, the locking actuator 320 in this embodiment includes a pin 321 and an elastic element 322. The pin 321 in this embodiment is provided with an arc-shaped protrusion 324. The magnetic actuator 310 in this embodiment includes a support frame 311, a sliding frame 312, and an attracted end 313 connected to the sliding frame 312. The sliding frame 312 in this embodiment is provided with a second inclined surface 314.

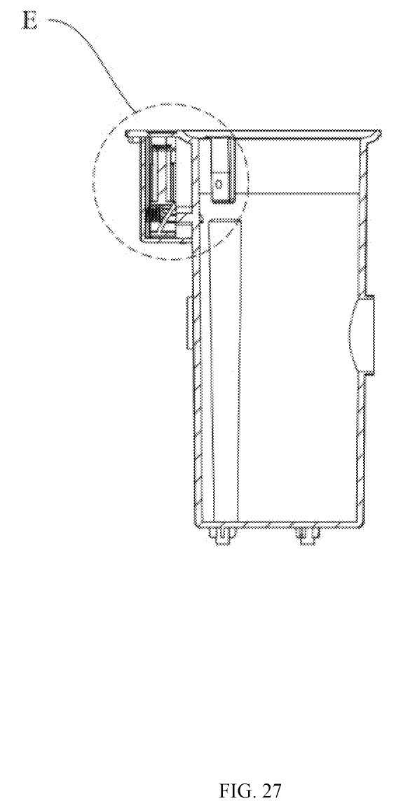

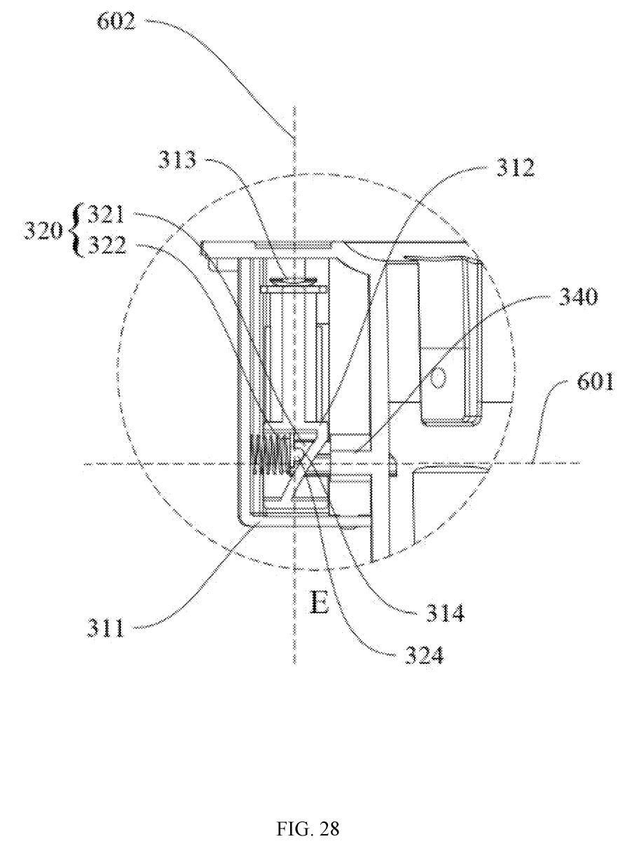

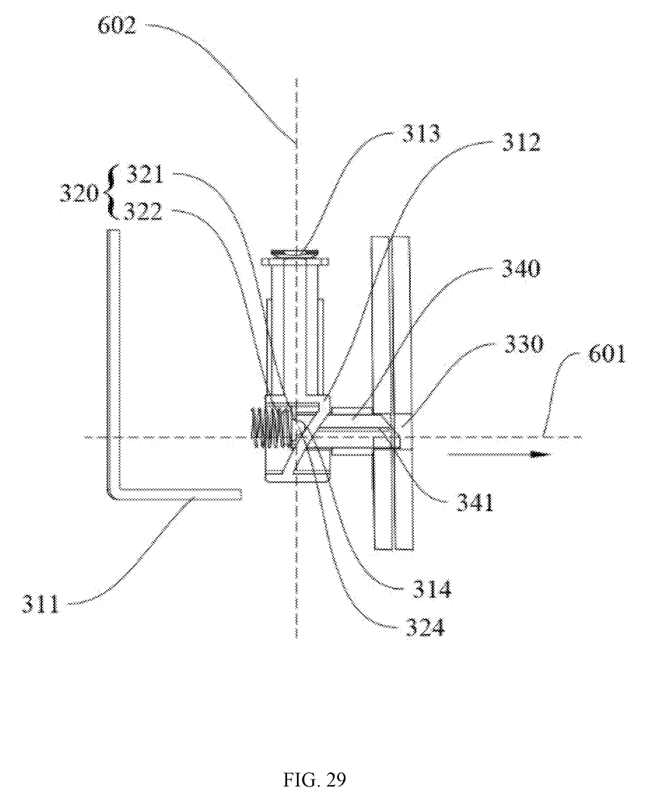

In combination with FIGS. 27, 28, and 29, the sliding frame 312 is slidably installed inside the support frame 311 along the second axis 602. The elastic element 322 is arranged at an end of the pin 321. The other end of the pin 321 facing the locking hole 330 is pushed by the elastic element 322 to pass through the sliding frame 312 and then be inserted into the locking hole 330 along the first axis 601, with the second inclined surface 314 abutted against the arc-shaped surface of the arc-shaped protrusion 324.

The outer surface of the pin 321 is provided with a plurality of ridges 341 along the first axis 601, and a directional tunnel hole 340 is provided between the pin 321 and the locking hole 330, which defines the direction in which the pin 321 is inserted into the locking hole 330.

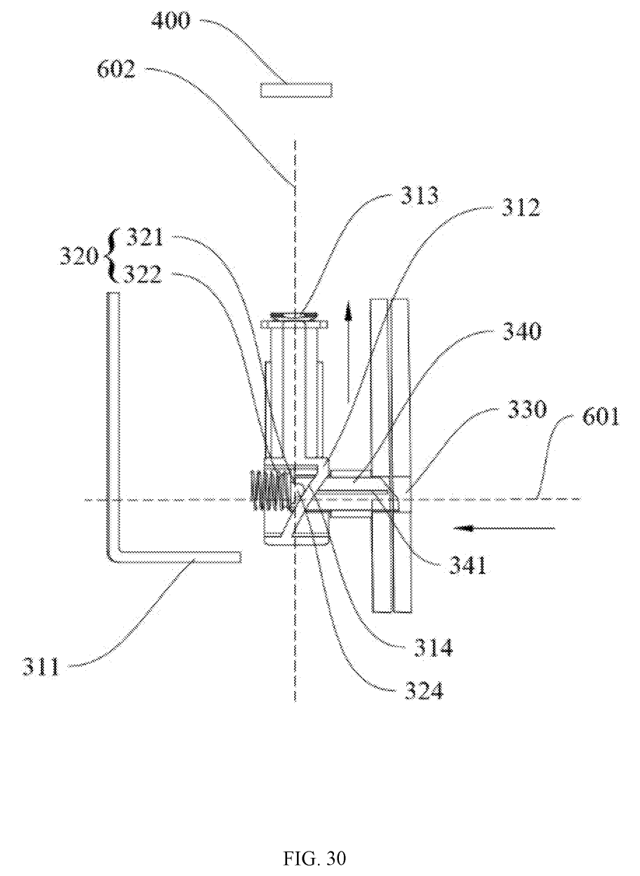

As shown in FIG. 30, when the unlocking part 400 is close to the unlocking area 500, or placed in the unlocking area 500, the sliding frame 312 slides along the second axis 602, allowing the second inclined surface 314 to push the arc-shaped protrusion 324 to drive the pin 321 to compress the elastic element 322 along the first axis 601, so as to drive the locking actuator 320 to disengage from the locking hole 330, thereby achieving the unlocking action.

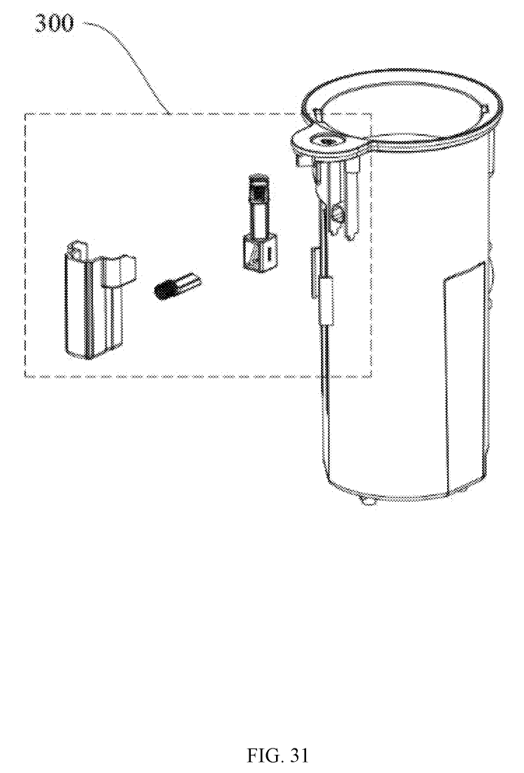

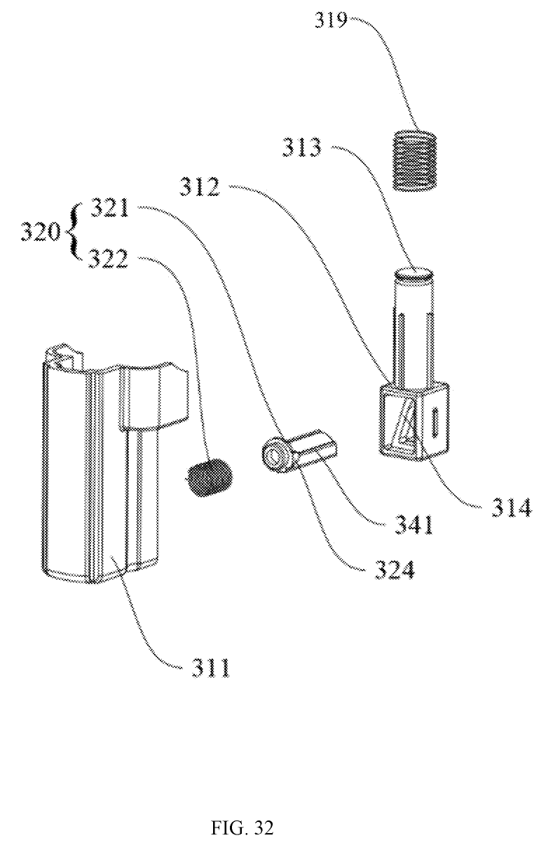

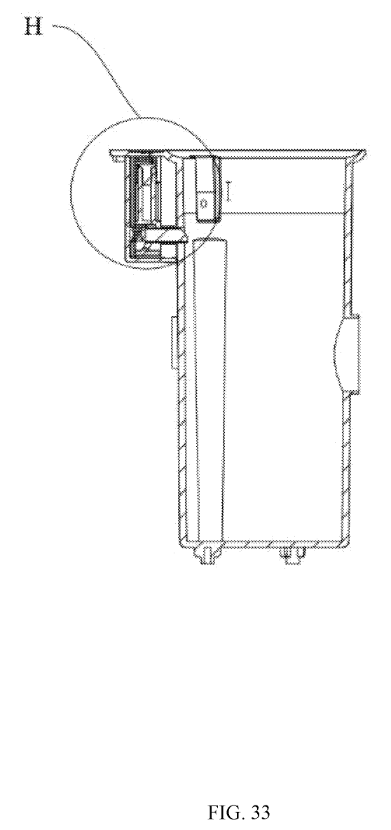

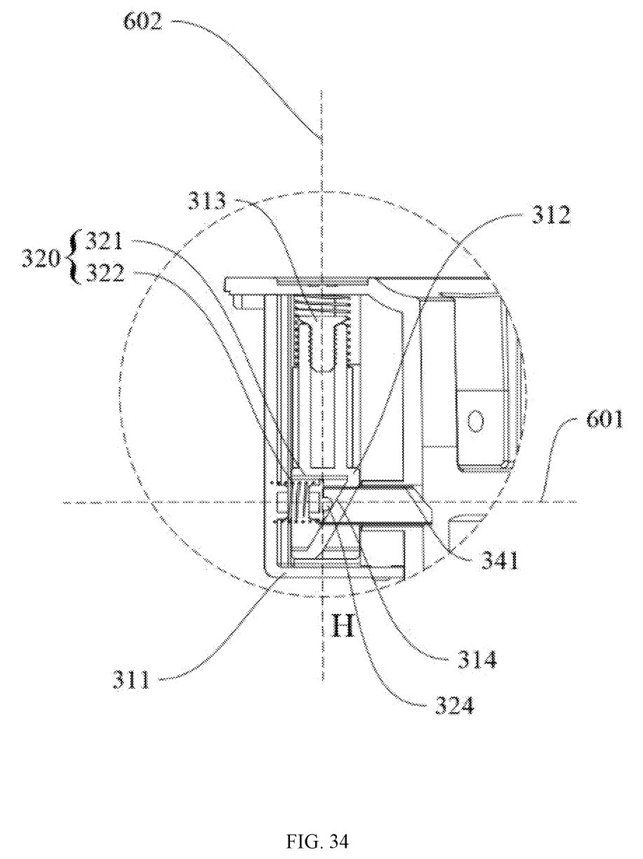

Further referring to FIGS. 31-34, the attracted end 313 of the sliding frame 312 in this embodiment is further provided with an additional spring structure 319. In this embodiment, when the unlocking part 400 is moved away from the unlocking area 500, the spring structure 319 facilitates downward sliding of the sliding frame 312 along the second axis 602, so as to drive the locking actuator 320 to be inserted into the locking hole 330, thereby achieving the relocking action.

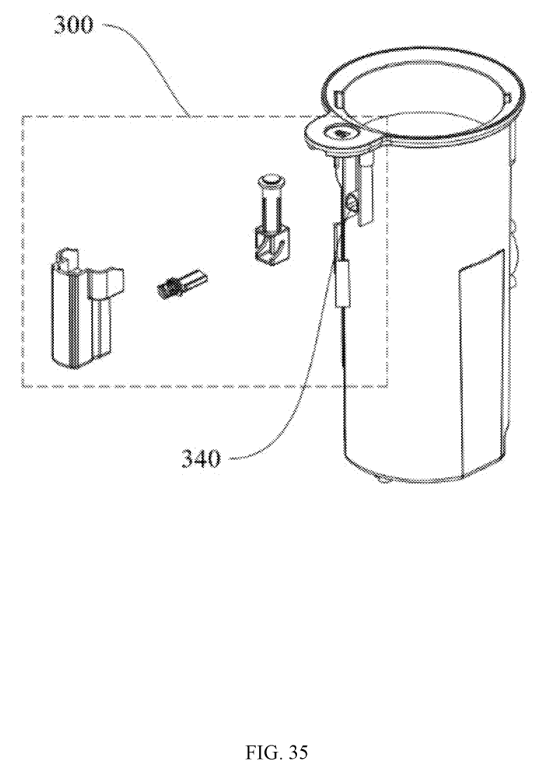

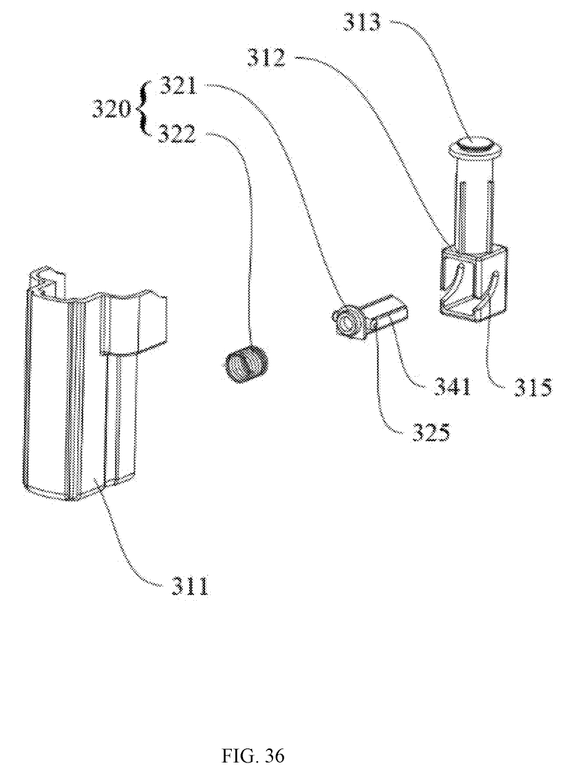

FIGS. 35 and 36 provide another locking mechanism 300 according to an embodiment of the present disclosure. The locking actuator 320 in this embodiment includes a pin 321 and an elastic element 322, and the magnetic actuator 310 includes a support frame 311, a sliding frame 312, and an attracted end 313 connected to the sliding frame 312. In this embodiment, the pin 321 is provided with a sliding shaft 325, and the sliding frame 312 is provided with an arc-shaped sliding groove 315.

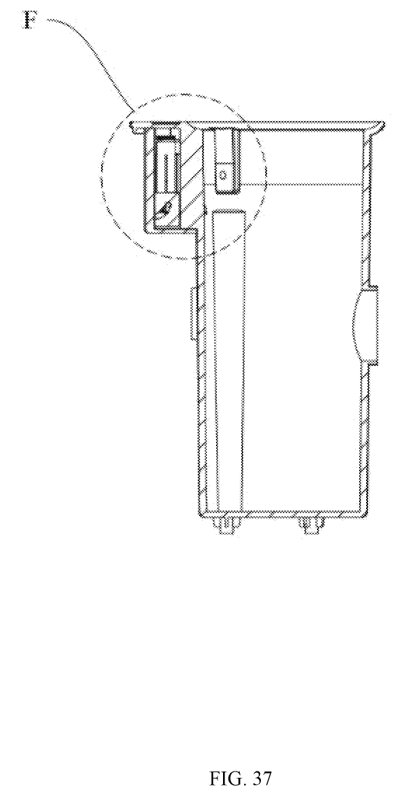

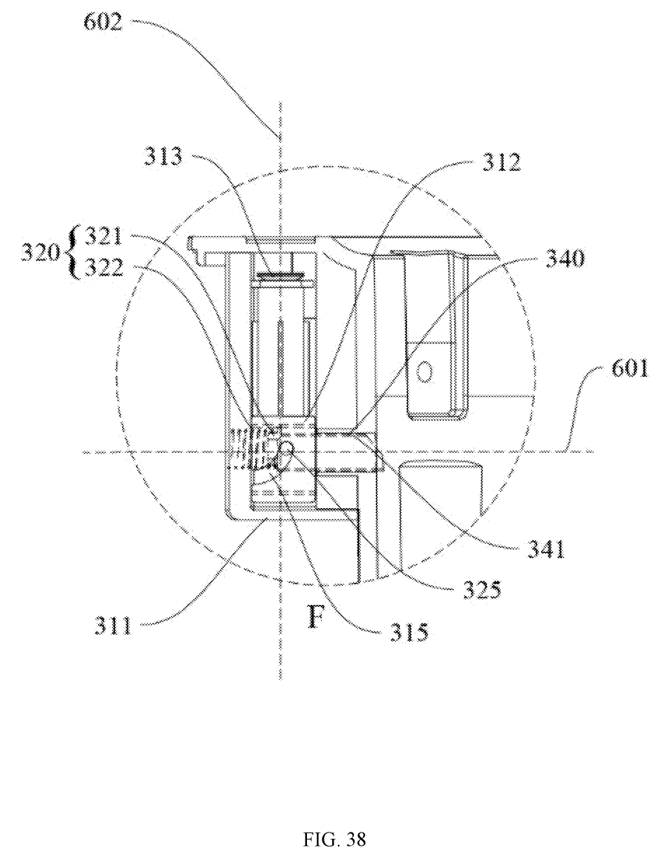

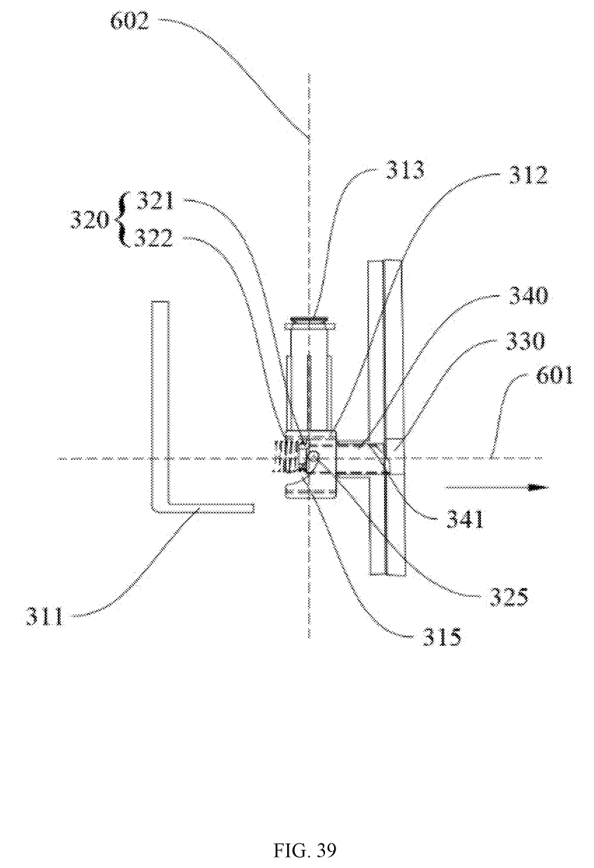

In conjunction with FIGS. 37, 38, and 39, in this embodiment, the sliding frame 312 is slidably installed inside the support frame 311 along the second axis 602, and the elastic element 322 is arranged at one end of the pin 321, the other end of the pin 321 facing the locking hole 330 is pushed by the elastic element 322 to pass through the sliding frame 312 and then be inserted into the locking hole 330 along the first axis 601, with the sliding shaft 325 located in the arc-shaped sliding groove 315.

In addition, the outer surface of the pin 321 in this embodiment is provided with a plurality of ridges 341 along the first axis 601, and a directional tunnel hole 340 is arranged between the pin 321 and the locking hole 330, which defines the direction in which the pin 321 is inserted into the locking hole 330.

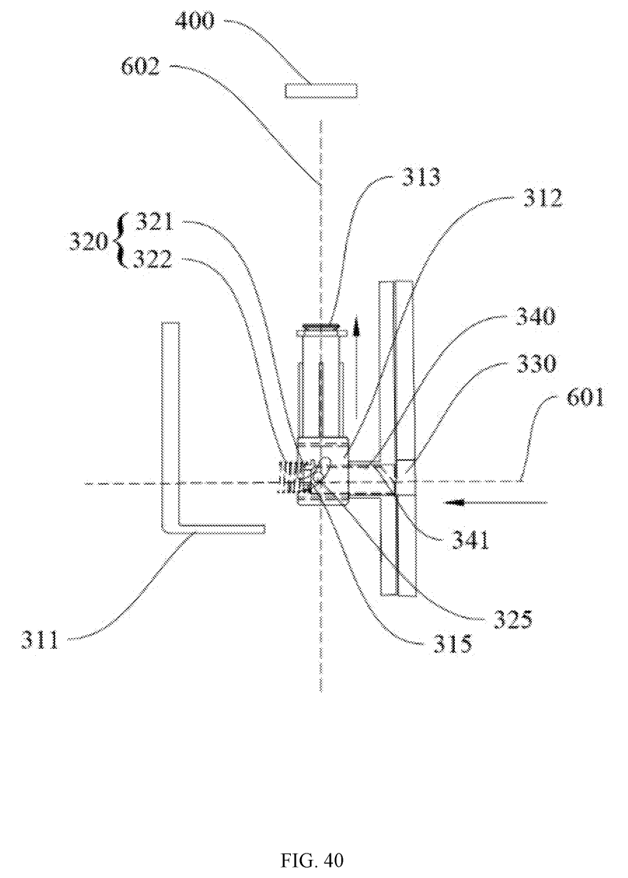

As shown in FIG. 40, when the unlocking part 400 is close to the unlocking area 500, or placed in the unlocking area 500, the sliding frame 312 slides along the second axis 602, allowing the sliding shaft 325 to slide in the arc-shaped sliding groove 315 to drive the pin 321 to compress the elastic element 322 along the first axis 601, so as to drive the locking actuator 320 to disengage from the locking hole 330, thereby achieving the unlocking action.

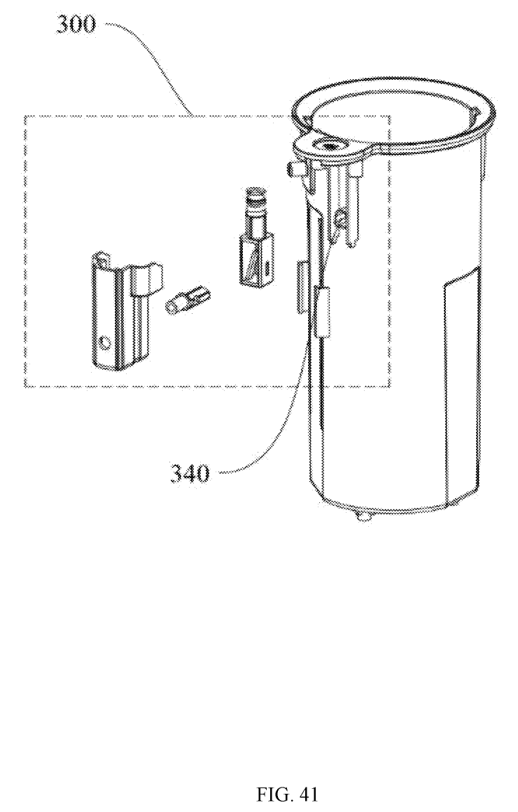

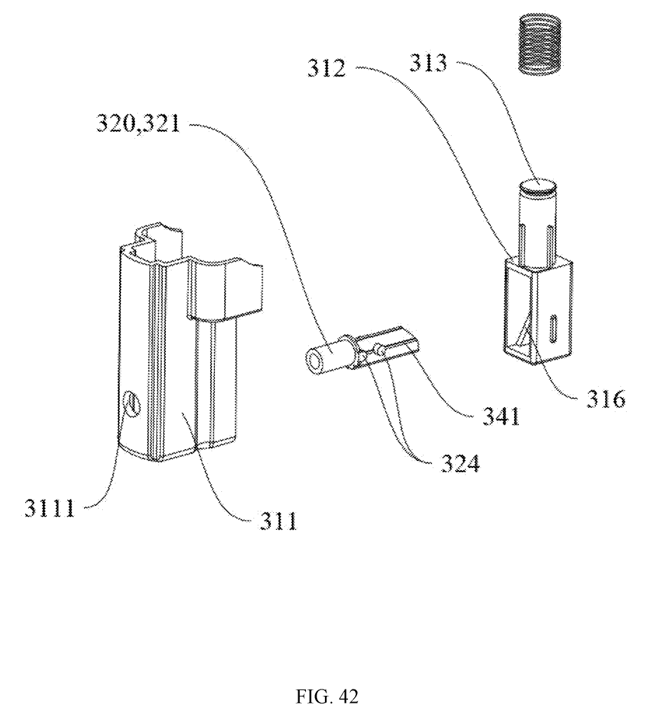

FIGS. 41 and 42 provide another locking mechanism 300 according to an embodiment of the present disclosure. The locking actuator 320 in this embodiment includes a pin 321 provided with arc-shaped protrusions 324 opposite to each other, with a sliding gap formed between the opposite arc-shaped surfaces of the arc-shaped protrusions 324. The magnetic actuator 310 in this embodiment includes a support frame 311, a sliding frame 312, and an attracted end 313 connected to the sliding frame 312. In this embodiment, the support frame 311 is provided with a directional hole 3111 corresponding to the pin 321, and the sliding frame 312 is provided with an inclined sliding bar 316.

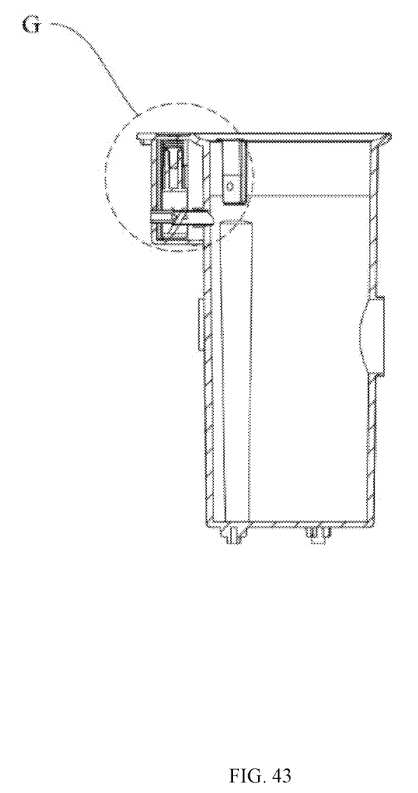

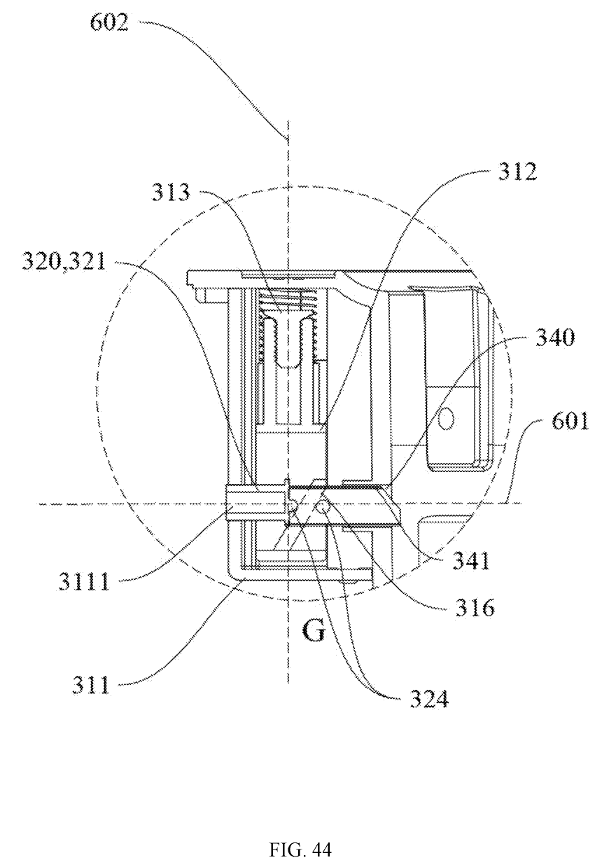

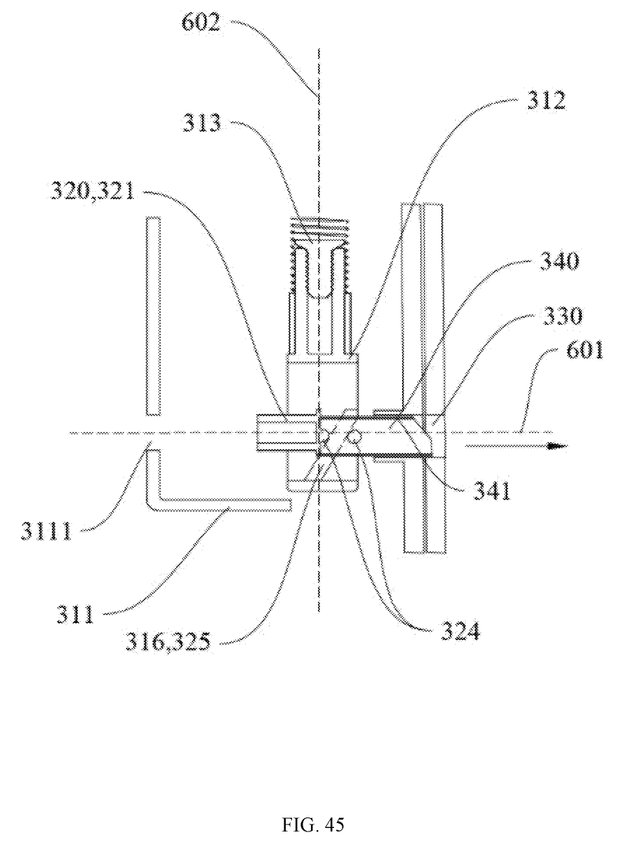

In conjunction with FIGS. 43, 44, and 45, the sliding frame 312 is slidably installed inside the support frame 311 along the second axis 602, one end of the pin 321 facing the locking hole 330 passes through the sliding frame 312 and then be inserted into the locking hole 330 along the first axis 601, and the inclined sliding bar 316 is located in the sliding gap. One end of the pin 321 away from the locking hole 330 is extended into the directional hole 3111, which can slide in the directional hole 3111.

In addition, the outer surface of the pin 321 in this embodiment is further provided with a plurality of ridges 341 along the first axis 601, and a directional tunnel hole 340 is arranged between the pin 321 and the locking hole 330, which defines the direction in which the pin 321 is inserted into the locking hole 330.

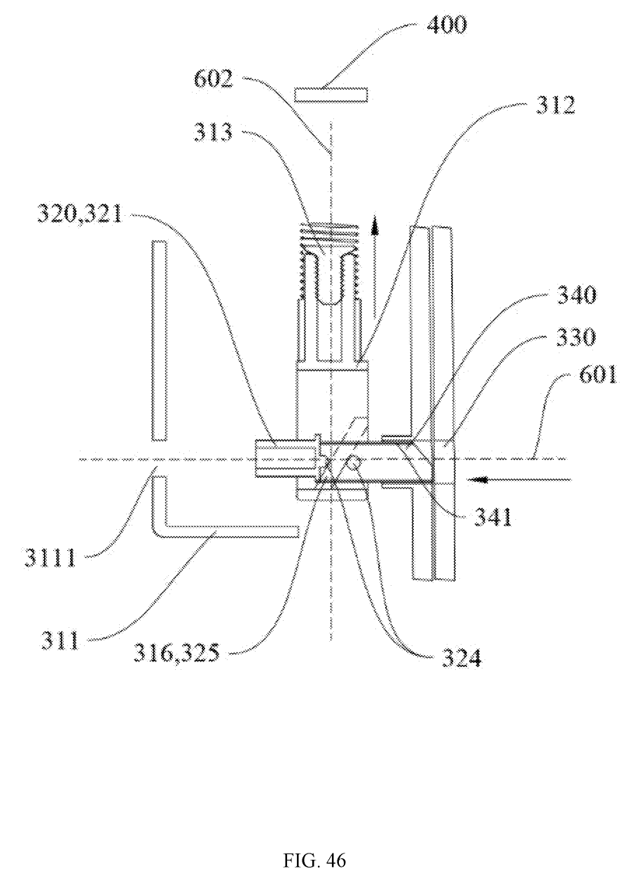

As shown in FIG. 46, when the unlocking part 400 is close to the unlocking area 500, or placed in the unlocking area 500, the sliding frame 312 slides along the second axis 602, allowing the inclined sliding bar 316 to slide in the sliding gap to push the pin 321 to slide along the first axis 601, so as to drive the locking actuator 320 to disengage from the locking hole 330, thereby achieving the unlocking action.

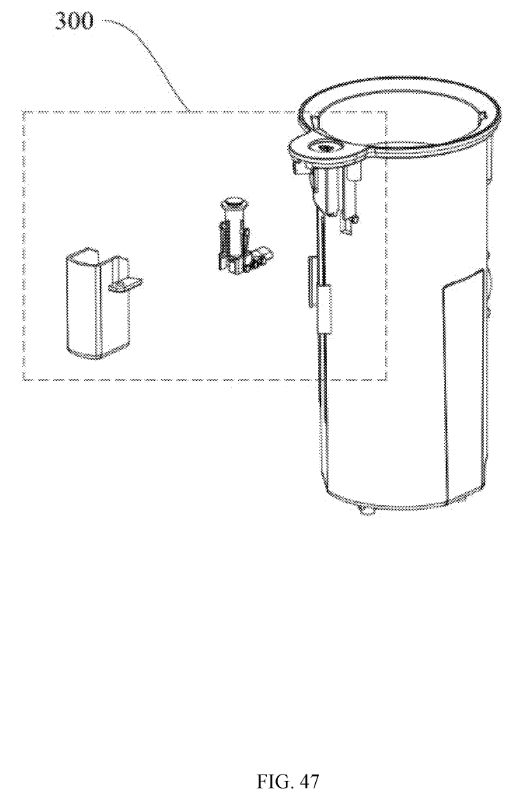

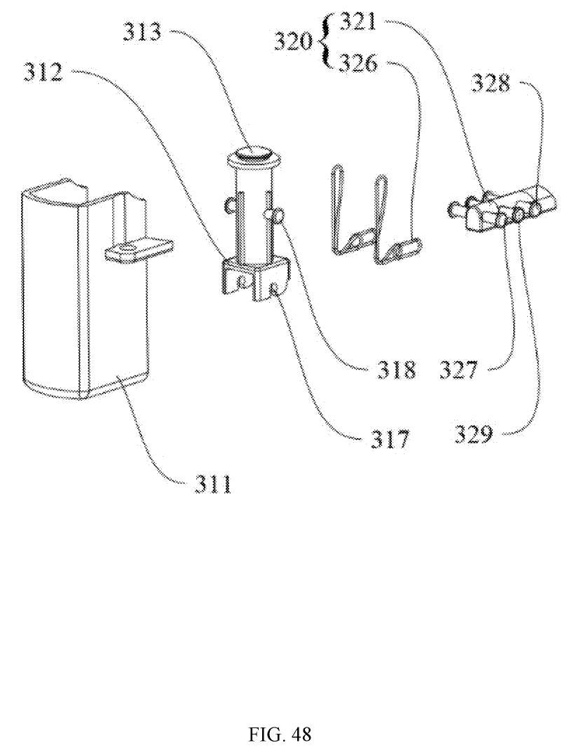

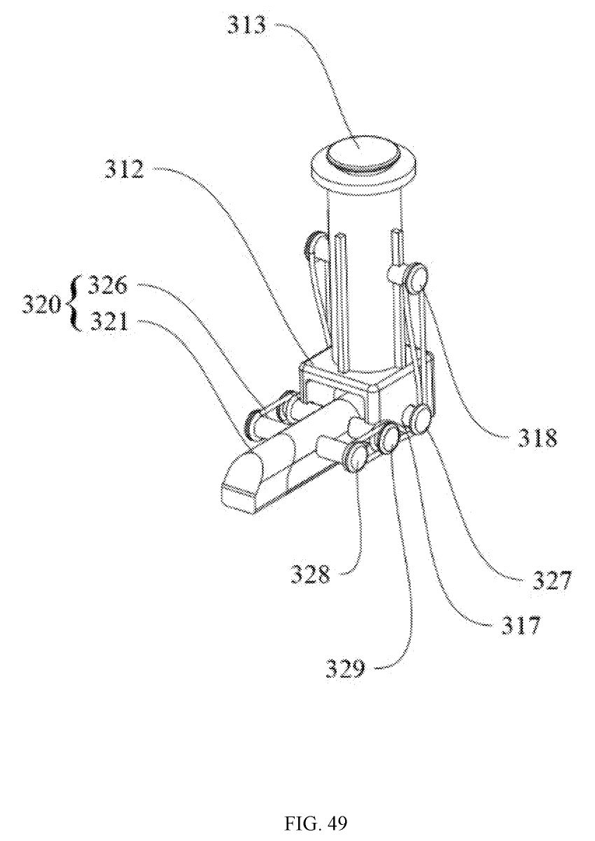

FIGS. 47 and 48 provide another locking mechanism 300 according to an embodiment of the present disclosure. In conjunction with FIG. 49, the locking actuator 320 in this embodiment includes a pin 321 and an elastic belt 326. The pin 321 in this embodiment is rotatably inserted into the locking hole 330. The proximal end of the pin 321 is provided with a rotating shaft 327, and the distal end of the pin 321 is provided with a second positioning portion 328 associated with the elastic belt 326. An intermediate shaft 329 is further arranged between the rotating shaft 327 and the second positioning portion 328.

The magnetic actuator 310 in this embodiment includes a support frame 311, a sliding frame 312, and an attracted end 313 connected to the sliding frame 312. The sliding frame 312 is provided with a shaft seat 317 for supporting the rotating shaft 327. The attracted end 313 is provided with a first positioning portion 318 associated with the elastic belt 326. The elastic belt 326 is in an annular shape, with two ends wound around the first positioning portion 318 and the second positioning portion 328, parallel belt body in the middle passing around the rotating shaft 327 on the same side, and the elastic belt 326 is wound around the intermediate shaft 329 for one turn.

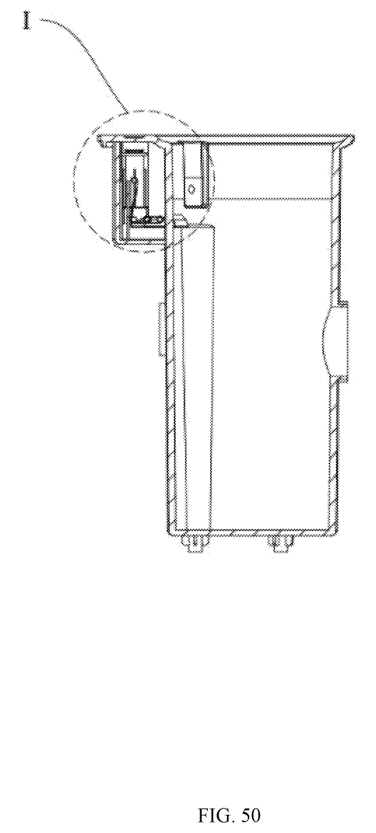

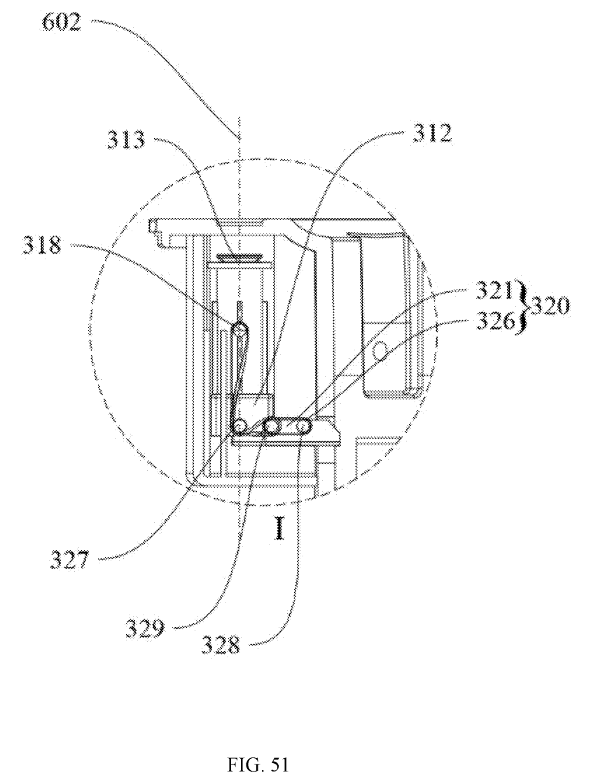

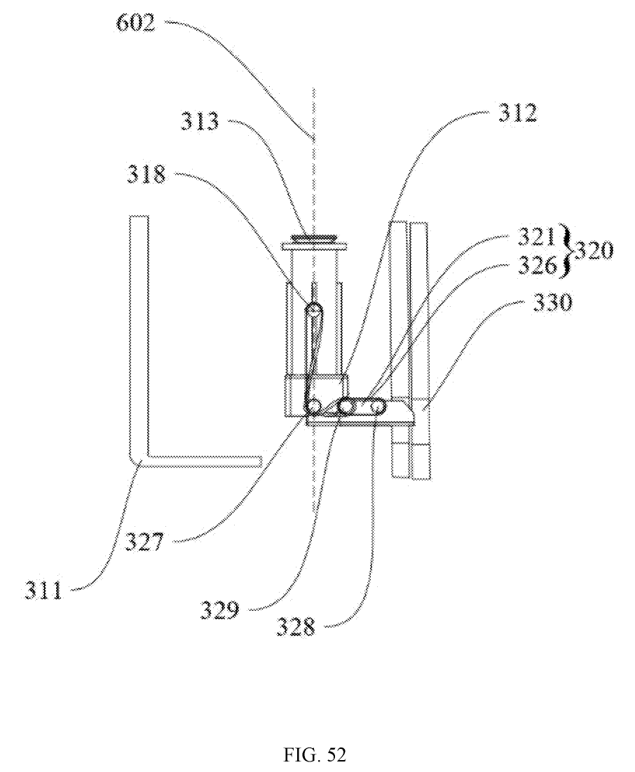

Referring to FIGS. 50, 51 and 52, the sliding frame 312 is slidably installed inside the support frame 311 along the second axis 602, one end of the pin 321 facing the locking hole 330 is extended through the sliding frame 312, and the elastic belt 326 is connected between the first positioning portion 318 and the second positioning portion 328 and passes around the rotating shaft 327 to stay in a stretched state.

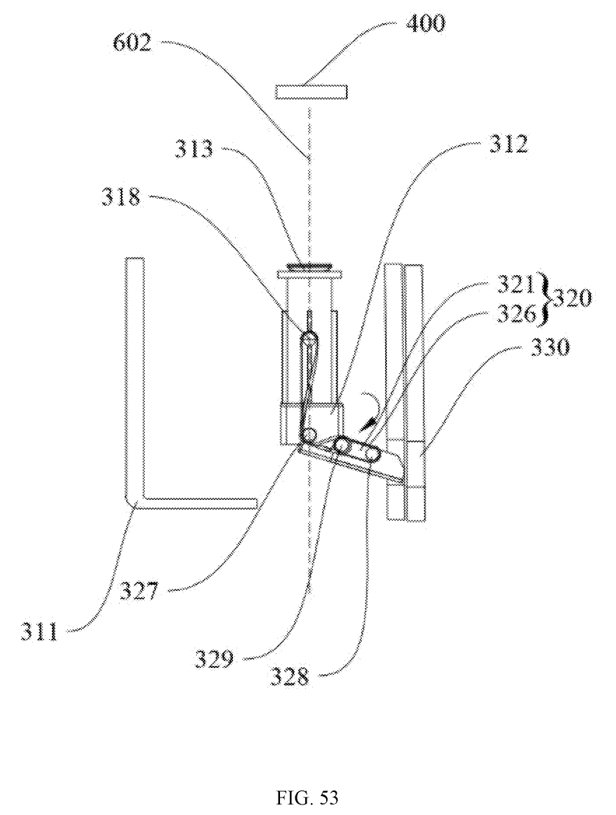

Further in conjunction with FIG. 53, when the unlocking part 400 is close to the unlocking area 500, or placed in the unlocking area 500, the sliding frame 312 slides upward along the second axis 602, allowing the elastic belt 326 to pull the pin 321 to rotate around the rotating shaft 327 and increase the angle between the first axis 601 and the second axis 602, so as to drive the locking actuator 320 to disengage from the locking hole 330, thereby achieving the unlocking action.

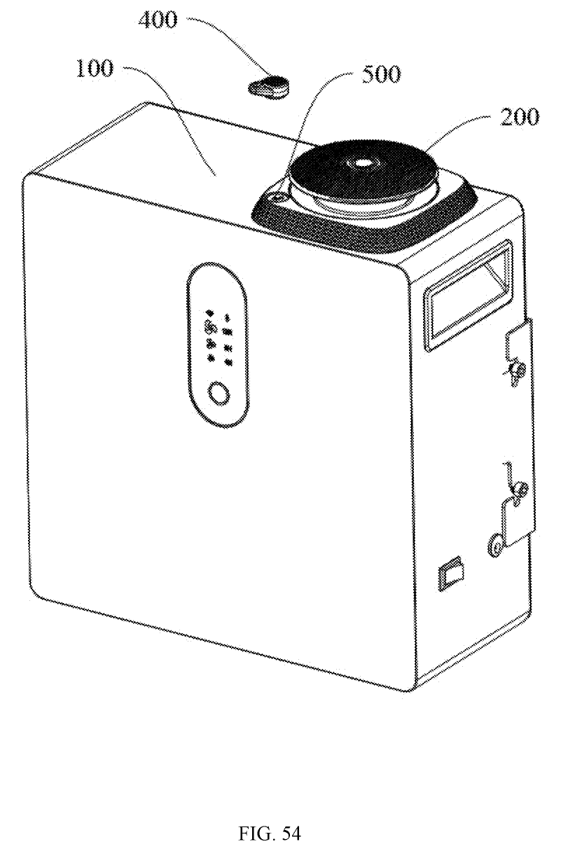

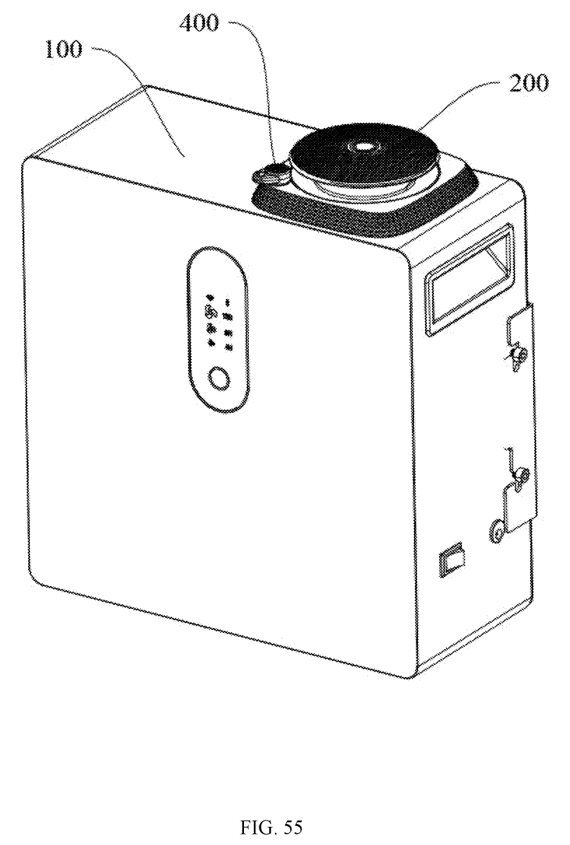

FIGS. 54 and 55 provide another inductive unlocking atomizer according to an embodiment of the present disclosure, which may have the locking mechanism 300 in any embodiment mentioned above. The unlocking area 500 in this embodiment protrudes from the upper surface of the housing 100. Such configuration in this embodiment can readily remove the unlocking part 400 from the unlocking area 500 for unlocking.

Obviously, the aforementioned embodiments of the present disclosure are merely examples provided to clearly illustrate the technical solutions of the present disclosure, and are not intended to limit the specific implementations of the present disclosure. Any modifications, equivalent substitutions, improvements, etc., made within the spirit and principles outlined in the claims of the present disclosure shall fall within the scope of protection of the claims of the present disclosure.

What is claimed is:

1. An inductive unlocking atomizer, comprising:
a housing;
an atomizing assembly positioned in the housing;
a locking mechanism configured for locking the atomizing assembly to the housing; and
a separate unlocking part configured for releasing locking of the locking mechanism via induction,
wherein the housing or the atomizing assembly is provided with an unlocking area for supporting the unlocking part during unlocking, and
the locking mechanism is configured to unlock the atomizing assembly from the housing, in response to the unlocking part being on or within the unlocking area, and further configured to relock the atomizing assembly to the housing, in response to the unlocking part being moved away from the unlocking area.

2. The inductive unlocking atomizer according to claim 1, wherein the unlocking part is configured to release the locking of the locking mechanism in a way of magnetic unlocking, radio frequency identification (RFID) unlocking, or Hall sensing unlocking.

3. The inductive unlocking atomizer according to claim 1, wherein the unlocking part is configured to release the locking of the locking mechanism in a way of magnetic unlocking, the unlocking area is in a form of a flat surface or a recess on the housing or the atomizing assembly for supporting the unlocking part during unlocking, and the locking mechanism is disposed adjacent to the unlocking part such that the locking mechanism and the unlocking part interact with each other.

4. The inductive unlocking atomizer according to claim 3, wherein the locking mechanism comprises a magnetic actuator and a locking actuator, and the locking actuator is driven by interaction between the unlocking part and the magnetic actuator to release the locking of the locking mechanism.

5. The inductive unlocking atomizer according to claim 4, wherein the locking actuator is a pin structure arranged inside the housing or the atomizing assembly, and the locking actuator is associated with a locking hole correspondingly provided in the atomizing assembly or the housing;
the locking actuator has a first axis, and the locking actuator is inserted into the locking hole along the first axis to lock the atomizing assembly to the housing; and
the magnetic actuator has a second axis not parallel to the first axis, and the magnetic actuator is driven by the unlocking part to move along the second axis and drive the locking actuator to disengage from the locking hole.

6. The inductive unlocking atomizer according to claim 5, wherein the second axis is perpendicular to the first axis.

7. The inductive unlocking atomizer according to claim 6,
wherein the locking actuator comprises:
a pin provided with a first inclined surface; and
an elastic element, configured to push the pin to insert into the locking hole along the first axis,
wherein the magnetic actuator comprises:
a support frame;
a sliding frame provided with a second inclined surface, disposed in the support frame and slidable along the second axis; and
an attracted end, connected to an end of the sliding frame extended out of the support frame, and
wherein the pin is extended through the sliding frame to allow the first inclined surface to be abutted against the second inclined surface, and the second inclined surface is configured to push the first inclined surface to drive the pin to compress the elastic element along the first axis, in response to the sliding frame sliding along the second axis.

8. The inductive unlocking atomizer according to claim 5,
wherein the locking actuator comprises:
a pin provided with a first inclined surface; and
an elastic element sleeved on the pin, configured to push the pin to insert into the locking hole along the first axis,
wherein the magnetic actuator comprises:
a support frame provided with a directional hole corresponding to the pin, wherein an end of the pin away from the locking hole is extended into the directional hole and slidable within the directional hole;
a sliding frame provided with a second inclined surface, disposed in the support frame and slidable along the second axis; and
an attracted end connected to the sliding frame, and
wherein an end of the pin facing the locking hole is extended through the sliding frame to allow the first inclined surface to be abutted against the second inclined surface, and the second inclined surface is configured to push the first inclined surface to drive the pin to compress the elastic element along the first axis, in response to the sliding frame sliding along the second axis.

9. The inductive unlocking atomizer according to claim 5,
wherein the locking actuator comprises:
a pin provided with an arc-shaped protrusion; and
an elastic element, configured to push the pin to insert into the locking hole along the first axis,
wherein the magnetic actuator comprises:
a support frame;
a sliding frame provided with a second inclined surface, disposed in the support frame and slidable along the second axis; and
an attracted end connected to the sliding frame, and
wherein an end of the pin facing the locking hole is extended through the sliding frame to allow an arc-shaped surface of the arc-shaped protrusion to be abutted against the second inclined surface, and the second inclined surface is configured to push the arc-shaped protrusion to drive the pin to compress the elastic element along the first axis, in response to the sliding frame sliding along the second axis.

10. The inductive unlocking atomizer according to claim 5,
wherein the locking actuator comprises:
a pin provided with a sliding shaft; and
an elastic element, configured to push the pin to insert into the locking hole along the first axis,
wherein the magnetic actuator comprises:
a support frame;
a sliding frame provided with an arc-shaped sliding groove, disposed in the support frame and slidable along the second axis; and
an attracted end connected to the sliding frame, and
wherein an end of the pin facing the locking hole is extended through the sliding frame to allow the sliding shaft to be positioned in the arc-shaped sliding groove, and the sliding shaft is configured to slide in the arc-shaped sliding groove to drive the pin to compress the elastic element along the first axis, in response to the sliding frame sliding along the second axis.

11. The inductive unlocking atomizer according to claim 5,
wherein the locking actuator comprises:
a pin inserted into the locking hole along the first axis, provided with arc-shaped protrusions opposite to each other, with a sliding gap formed between opposite arc-shaped surfaces of the arc-shaped protrusions;
wherein the magnetic actuator comprises:
a support frame;
a sliding frame provided with an inclined sliding bar, disposed in the support frame and slidable along the second axis; and
an attracted end connected to the sliding frame, and
wherein an end of the pin facing the locking hole is extended through the sliding frame to allow the inclined sliding bar to be positioned in the sliding gap, and the inclined sliding bar is configured to slide in the sliding gap to drive the pin to move along the first axis, in response to the sliding frame sliding along the second axis.

12. The inductive unlocking atomizer according to claim 11, wherein the support frame is provided with a directional hole corresponding to the pin, and an end of the pin away from the locking hole is extended into the directional hole and slidable in the directional hole.

13. The inductive unlocking atomizer according to claim 5,
wherein the locking actuator comprises:
a pin rotatably inserted into the locking hole, provided with a rotating shaft at a proximal end of the pin and a second positioning portion at a distal end of the pin; and
an elastic belt associated with the second positioning portion,
wherein the magnetic actuator comprises:
a support frame;
a sliding frame provided with a shaft seat for supporting the rotating shaft, disposed in the support frame and slidable along the second axis; and
an attracted end connected to the sliding frame, wherein the sliding frame or the attracted end is provided with a first positioning portion associated with the elastic belt, and
wherein an end of the pin facing the locking hole is extended through the sliding frame, and the elastic belt is in a stretched state by connecting between the first positioning portion and the second positioning portion and passing around the rotating shaft, the elastic belt is configured to drive the pin to rotate around the rotating shaft to increase an angle between the first axis and the second axis, in response to the sliding frame sliding along the second axis.

14. The inductive unlocking atomizer according to claim 13, wherein the elastic belt is in a form of an annular shape, with two ends of the elastic belt respectively wound around the first positioning portion and the second positioning portion, and a parallel portion in a middle portion passing around the rotating shaft on the same side.

15. The inductive unlocking atomizer according to claim 14, wherein an intermediate shaft is further arranged between the rotating shaft and the second positioning portion, and the elastic belt is wound around the intermediate shaft for at least one turn.

16. The inductive unlocking atomizer according to claim 5, wherein the locking actuator comprises a pin, and a directional tunnel hole is arranged between the pin and the locking hole, the directional tunnel hole is configured to define a direction in which the pin is inserted into the locking hole.

17. The inductive unlocking atomizer according to claim 16, wherein an outer surface of the pin is provided with one or more ridges distributed along the first axis.

18. The inductive unlocking atomizer according to claim 3, wherein the unlocking part has a magnetic attracting end, the locking mechanism is provided with a magnetic attracted end, and the attracting end is configured to drive the attracted end to move to release the locking of the locking mechanism, in response to the unlocking part being placed in the unlocking area.

19. The inductive unlocking atomizer according to claim 1, wherein the atomizing assembly comprises an atomizing base, and an aroma liquid container connected to the atomizing base and sealed inside the housing via the atomizing base, and the locking mechanism is configured to lock the atomizing base to the housing.

20. The inductive unlocking atomizer according to claim 1, wherein the housing comprises a power chamber and an atomizing chamber, the power chamber and the atomizing chamber are separated from each other,
a power supply component and an air source component are arranged in the power chamber,
the atomizing assembly is arranged in the atomizing chamber, and
the air source component is extended into the atomizing chamber through an air supply tube to supply high-pressure gas to the atomizing assembly.

21. The inductive unlocking atomizer according to claim 1, wherein the unlocking area is protruded from an upper surface of the housing.
